(19) United States
(12) Patent Application Publication
(10) Pub. No.: US 2023/0303693 A1
(43) Pub. Date: Sep. 28, 2023

(54) BISPECIFIC ANTIBODY AGAINST CD3 AND CD20 IN COMBINATION THERAPY FOR TREATING DIFFUSE LARGE B-CELL LYMPHOMA

(71) Applicant: GENMAB A/S, Copenhagen V (DK)

(72) Inventors: Christopher W. L. CHIU, Warren, NJ (US); Mariana Cota STIRNER, Summit, NJ (US); Minh H. DINH, Northbrook, IL (US); IIiana Edith SZAFER-GLUSMAN, San Jose, CA (US)

(21) Appl. No.: 18/160,391

(22) Filed: Jan. 27, 2023

Related U.S. Application Data
(60) Provisional application No. 63/304,422, filed on Jan. 28, 2022.

(51) Int. Cl. C07K 16/28; A61P 35/00; A61K 47/68; A61K 39/395; A61K 31/675; A61K 31/704; A61K 31/573

(52) U.S. Cl. CPC C07K 16/2809; A61P 35/00; A61K 47/6811; A61K 47/6867; A61K 39/3955; A61K 31/675; A61K 31/704; A61K 31/573; C07K 16/2887; A61K 2039/507

(57) ABSTRACT

Provided are methods of clinical treatment of diffuse large B-cell lymphoma (DLBCL) (e.g., previously untreated DLBCL) in human subjects using a bispecific antibody which binds to CD3 and CD20 in combination with Pola-R-CHP (polatuzumab vedotin, rituximab, cyclophosphamide, doxorubicin, and prednisone).

### FIELD

The present invention relates to bispecific antibodies targeting both CD3 and CD20 and the use of such antibodies in combination with polatuzumab vedotin and R-CHP rituximab, cyclophosphamide, doxorubicin and prednisone or an equivalent thereof; e.g. prednisolone regimen for the treatment of diffuse large B-cell lymphoma (DLBCL), for example, previously untreated DLBCL; e.g. in subjects with an International Prognostic Index (IPI) score of 2-5. Advantageous treatment regimens are also provided.

### BACKGROUND

DLBCL is the most common non-Hodgkin lymphoma (NHL), and the standard first-line therapy is R-CHOP. The cure rate of this combination for the overall population of newly-diagnosed DLBCL is between 60% and 70% (Sehn et al., Blood 2007; 109:1867-61). Attempts to improve upon outcomes of first-line therapy, including intensification of dose and addition of other agents to intensify the regimen, have failed to provide sufficient evidence to alter standard of care.

Risk factors impacting rates of CR to first-line treatment, disease relapse, and OS are included in the International Prognostic Index (IPI) or Revised-IPI (R-IPI): age>60 years, ECOG>1 or KPS<60, LDH>ULN; extranodal disease>1 (2 or more), and disease Stage 3 or 4 (Project et al., N Engl J Med 1993; 329:987-994; Sehn et al., supra). While patients in the good risk group (1-2 IPI factors) have a 4-year PFS of 80% following standard first-line R-CHOP, the 45% of patients in the poor risk (high risk) group (3-5 IPI factors) only achieve a 4-year PFS and OS of 55% (Sehn et al., supra).

Given the limited efficacy and long-term response of poor risk subjects to currently available treatments, novel and effective treatments are needed.

### SUMMARY

Provided herein are methods of treating human subjects who have DLBCL, for example, previously untreated DLBCL (e.g., DLBCL with International Prognostic Index (IPI) score of 2-5)), by administering a bispecific antibody which binds to CD3 and CD20 in combination with polatuzumab vedotin, rituximab and cyclophosphamide, doxorubicin and prednisone or an equivalent thereof, e.g. prednisolone (R-CHP), in particular, advantageous clinical treatment regimens.

In one aspect, provided herein is a method of treating diffuse large B-cell lymphoma (DLBCL) in a human subject, the method comprising administering to the subject the combination of epcoritamab with polatuzumab vedotin and rituximab, cyclophosphamide, doxorubicin and prednisone or an equivalent thereof, e.g. prednisolone (R-CHP), e.g., the method comprising administering to the subject an effective amount of polatuzumab vedotin, rituximab, cyclophosphamide, doxorubicin and a glucocorticoid, such as an effective amount of each of polatuzumab vedotin, rituximab, cyclophosphamide, doxorubicin and prednisone or an equivalent thereof, e.g. prednisolone.

In one aspect, provided herein is a method of treating diffuse large B-cell lymphoma (DLBCL) in a human subject, the method comprising administering to the subject a bispecific antibody and an effective amount of polatuzumab vedotin, rituximab, cyclophosphamide, doxorubicin and prednisone or an equivalent thereof, e.g. prednisolone, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose of 24 mg or 48 mg, and wherein polatuzumab vedotin, rituximab, cyclophosphamide, doxorubicin and prednisone or an equivalent thereof, e.g. prednisolone, and the bispecific antibody are administered in 21-day cycles.

In some embodiments, the bispecific antibody is administered at a dose of (or a dose of about) 24 mg. In some embodiments, the bispecific antibody is administered at a dose of (or a dose of about) 48 mg.

In one embodiment, the bispecific antibody is administered once every week at a dose of 24 mg or 48 mg (weekly administration/dose), e.g., for three and one-third 21-day cycles (i.e., day 15 of cycle 1 and days 1, 8, and 15 of cycle 2-4). In some embodiments, the bispecific antibody is administered once every three weeks after the weekly administration, e.g., for at least two or four 21-day cycles, such as for four 21-day cycles. In a further embodiment, a priming dose (e.g., 0.16 mg or about 0.16 mg) of the bispecific antibody is administered two weeks prior to administering the first weekly dose of 24 mg or 48 mg. In some embodiments, after administering the priming dose and prior to administering the weekly dose of 24 mg or 48 mg, an intermediate dose (e.g., 0.8 mg or about 0.8 mg) of the bispecific antibody is administered. In some embodiments, the priming dose is administered one week before the intermediate dose, and the intermediate dose is administered one week before the first weekly dose of 24 mg or 48 mg.

In some embodiments, polatuzumab vedotin is administered in a 21-day cycle once every three weeks, e.g., for at least six 21-day cycles, such as for six 21-day cycles. In some embodiments, polatuzumab vedotin is administered at a dose of 1.8 mg/kg.

In some embodiments, rituximab is administered in a 21-day cycle once every three weeks, e.g., for six or eight 21-day cycles. In some embodiments, rituximab is administered at a dose of 375 mg/m².

In some embodiments, cyclophosphamide is administered in a 21-day cycle once every three weeks, e.g., for six or eight 21-day cycles. In some embodiments, cyclophosphamide is administered at a dose of 750 mg/m².

In some embodiments, doxorubicin is administered in a 21-day cycle once every three weeks, e.g., for six or eight 21-day cycles. In some embodiments, doxorubicin is administered at a dose of 50 mg/m².

In some embodiments, prednisone or the equivalent thereof is administered once a day from day 1 to day 5 of the 21-day cycles, e.g., for six or eight 21-day cycles. In some embodiments, prednisone or the equivalent thereof; e.g. prednisolone, is administered at a dose of 100 mg/day.

In some embodiments, polatuzumab vedotin, rituximab, cyclophosphamide, doxorubicin, prednisone, and the bispecific antibody are administered on the same day (e.g., on day 1 of cycles 1-6

In some embodiments, administration is performed in 21-day cycles, wherein
(a) the bispecific antibody is administered as follows:
   (i) in cycle 1, a priming dose of 0.16 mg is administered on day 1, an intermediate dose of 0.8 mg is administered on day 8, and a dose of 24 mg is administered on day 15;
   (ii) in cycle 2-4, a dose of 24 mg is administered on days 1, 8, and 15;
   (iii) in cycles 5-8, a dose of 24 mg is administered on day 1;
(b) polatuzumab vedotin, rituximab, cyclophosphamide and doxorubicin are administered on day 1 in cycles 1-6; and
(c) prednisone or the equivalent thereof; e.g. prednisolone, is administered on days 1-5 in cycles 1-6.

In some embodiments, administration is performed in 21-day cycles, wherein
(a) the bispecific antibody is administered as follows:
   (i) in cycle 1, a priming dose of 0.16 mg is administered on day 1, an intermediate dose of 0.8 mg is administered on day 8, and a dose of 48 mg is administered on day 15;
   (ii) in cycle 2-4, a dose of 48 mg is administered on days 1, 8, and 15;
   (iii) in cycles 5-8, a dose of 48 mg is administered on day 1;
(b) polatuzumab vedotin, rituximab, cyclophosphamide and doxorubicin are administered on day 1 in cycles 1-6; and
(c) prednisone or the equivalent thereof; e.g. prednisolone is administered on days 1-5 in cycles 1-6.

In some embodiments, the bispecific antibody is administered subcutaneously. In some embodiments, polatuzumab vedotin is administered intravenously. In some embodiments, rituximab is administered intravenously. In some embodiments, cyclophosphamide is administered intravenously. In a further embodiment, doxorubicin is administered intravenously. In some embodiments, prednisone is administered intravenously or orally.

In some embodiments, the bispecific antibody, polatuzumab vedotin, rituximab, cyclophosphamide and doxorubicin are administered sequentially.

In some embodiments, the DLBCL is with histologically confirmed CD20+ disease. In some embodiments, the DLBCL is B cell lymphoma with MYC and Bcl-2 and/or Bcl-6 translocations (double-hit or triple-hit DLBCL). In some embodiments, the DLBCL is follicular lymphoma Grade 3B. In some embodiments, the subject has an International Prognostic Index (IPI) score or Revised IPI score of 2-5, such as a score of 2, 3, 4 or 5.

In some embodiments, the subject treated with the methods described herein achieves a complete response (CR), a partial response (PR), or stable disease (SD), e.g., as defined by the Lugano criteria or LYRIC.

In some embodiments, the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and the second antigen-binding region comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In some embodiments, the first antigen-binding region of the bispecific antibody comprises a VH region comprising the amino acid sequence of SEQ ID NO: 6, and the VL region comprising the amino acid sequence of SEQ ID NO: 7; and the second antigen-binding region comprises a VH region comprising the amino acid sequence of SEQ ID NO: 13, and the VL region comprising the amino acid sequence of SEQ ID NO: 14.

In some embodiments, the first binding arm of the bispecific antibody is derived from a humanized antibody, preferably from a full-length IgG1,λ (lambda) antibody (e.g., SEQ ID NO: 22). In some embodiments, the second binding arm of the bispecific antibody is derived from a human antibody, preferably from a full-length IgG1,κ (kappa) antibody (e.g., SEQ ID NO: 23). In some embodiments, the bispecific antibody is a full-length antibody with a human IgG1 constant region.

In some embodiments, the bispecific antibody comprises an inert Fc region, for example, an Fc region in which the amino acids in the positions corresponding to positions L234, L235, and D265 in the human IgG1 heavy chain constant region of SEQ ID NO: 15 are F, E, and A, respectively. In some embodiments, the bispecific antibody comprises substitutions which promote bispecific antibody formation, for example, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In some embodiments, the bispecific antibody has both an inert Fc region (e.g., substitutions at L234, L235, and D265 (e.g., L234F, L235E, and D265A)) and substitutions which promote bispecific antibody formation (e.g., F405L and K409R). In a further embodiment, the bispecific antibody comprises heavy chain constant regions comprising the amino acid sequences of SEQ ID NOs: 19 and 20.

In some embodiments, the bispecific antibody comprises a first heavy chain and a first light chain comprising (or consisting of) the amino acid sequences set forth in SEQ ID NOs: 24 and 25, respectively, and a second heavy chain and a second light chain comprising (or consisting of) the amino acid sequences set forth in SEQ ID NOs: 26 and 27, respectively. In some embodiments, the bispecific antibody is epcoritamab, or a biosimilar thereof.

### BRIEF DESCRIPTION OF THE DRAWINGS

FIGS. 1A-1C are graphs showing the minimal effects of CHP components on DuoBody® CD3xCD20-induced T-cell-mediated cytotoxicity. FIG. 1A: DuoBody® CD3xCD20+cyclophosphamide, FIG. 1B: DuoBody® CD3xCD20+doxorubicin, FIG. 1C: DuoBody® CD3xCD20+prednisone. Left panels show DuoBody® CD3xCD20 dose-response curves for one representative donor. Right panels show the results for 4 donors, at 333 ng/mL DuoBody® CD3xCD20.

FIG. 2 is a schematic of the overall clinical trial design.

### DETAILED DESCRIPTION

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized (see, e.g., Fundamental Immunology Ch. 7 (Paul, W., ed., 2nd ed. Raven Press, N.Y. (1989)). Briefly, each heavy chain typically is comprised of a heavy chain variable region (abbreviated herein as VH or VH) and a heavy chain constant region (abbreviated herein as CH or C_{H}). The heavy chain constant region typically is comprised of three domains, CH1, CH2, and CH3. The hinge region is the region between the CH1 and CH2 domains of the heavy chain and is highly flexible. Disulfide bonds in the hinge region are part of the interactions between two heavy chains in an IgG molecule. Each light chain typically is comprised of a light chain variable region (abbreviated herein as VL or V_{L}) and a light chain constant region (abbreviated herein as CL or C_{L}). The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J Mol Biol 1987; 196:90117). Unless otherwise stated or contradicted by context, CDR sequences herein are identified according to IMGT rules (Brochet X., Nucl Acids Res 2008; 36:W503-508; Lefranc M P., Nucl Acids Res 1999; 27:209-12; www.imgt.org/). Unless otherwise stated or contradicted by context, reference to amino acid positions in the constant regions is according to the EU-numbering (Edelman et al., PNAS. 1969; 63:78-85; Kabat et al., Sequences of Proteins of Immunological Interest, Fifth Edition. 1991 NIH Publication No. 91-3242). For example, SEQ ID NO: 15 sets forth amino acids positions 118-447, according to EU numbering, of the IgG1 heavy chain constant region.

The term “amino acid corresponding to position . . . ” as used herein refers to an amino acid position number in a human IgG1 heavy chain. Corresponding amino acid positions in other immunoglobulins may be found by alignment with human IgG1. Thus, an amino acid or segment in one sequence that “corresponds to” an amino acid or segment in another sequence is one that aligns with the other amino acid or segment using a standard sequence alignment program such as ALIGN, ClustalW or similar, typically at default settings and has at least 50%, at least 80%, at least 90%, or at least 95% identity to a human IgG1 heavy chain. It is within the ability of one of ordinary skill in the art to align a sequence or segment in a sequence and thereby determine the corresponding position in a sequence to an amino acid position according to the present invention.

The term “antibody” (Ab) as used herein in the context of the present invention refers to an immunoglobulin molecule which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The term antibody, unless specified otherwise, also encompasses polyclonal antibodies, monoclonal antibodies (mAbs), antibody-like polypeptides, chimeric antibodies and humanized antibodies An antibody as generated can possess any isotype.

The term “antibody fragment” or “antigen-binding fragment” as used herein refers to a fragment of an immunoglobulin molecule which retains the ability to specifically bind to an antigen, and can be generated by any known technique, such as enzymatic cleavage, peptide synthesis, and recombinant techniques. Examples of antibody fragments include (i) a Fab′ or Fab fragment, a monovalent fragment consisting of the VL, VH, CL and CH1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 1989; 341: 54446), which consists essentially of a VH domain and also called domain antibodies (Holt et al; Trends Biotechnol 2003; 21:484-90); (vi) camelid or nanobodies (Revets et al; Expert Opin Biol Ther 2005; 5:111-24) and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see, e.g., Bird et al., Science 1988; 242:42326 and Huston et al., PNAS 1988; 85:587983). Such single chain antibodies are encompassed within the term antibody fragment unless otherwise noted or clearly indicated by context.

The term “antibody-binding region” or “antigen-binding region” as used herein refers to the region which interacts with the antigen and comprises both the VH and the VL regions. The term antibody when used herein refers not only to monospecific antibodies, but also multispecific antibodies which comprise multiple, such as two or more, e.g., three or more, different antigen-binding regions. The term antigen-binding region, unless otherwise stated or clearly contradicted by context, includes fragments of an antibody that are antigen-binding fragments, i.e., retain the ability to specifically bind to the antigen.

As used herein, the term “isotype” refers to the immunoglobulin class (for instance IgG1, IgG2, IgG3, IgG4, IgD, IgA, IgE, or IgM) that is encoded by heavy chain constant region genes. When a particular isotype, e.g., IgG1, is mentioned, the term is not limited to a specific isotype sequence, e.g., a particular IgG1 sequence, but is used to indicate that the antibody is closer in sequence to that isotype, e.g. IgG1, than to other isotypes. Thus, e.g., an IgG1 antibody may be a sequence variant of a naturally-occurring IgG1 antibody, which may include variations in the constant regions.

The term “bispecific antibody” or “bs” or “bsAb” as used herein refers to an antibody having two different antigen-binding regions defined by different antibody sequences. A bispecific antibody can be of any format.

The terms “half molecule”, “Fab-arm”, and “arm”, as used herein, refer to one heavy chain-light chain pair.

When a bispecific antibody is described as comprising a half-molecule antibody “derived from” a first parental antibody, and a half-molecule antibody “derived from” a second parental antibody, the term “derived from” indicates that the bispecific antibody was generated by recombining, by any known method, said half-molecules from each of said first and second parental antibodies into the resulting bispecific antibody. In this context, “recombining” is not intended to be limited by any particular method of recombining and thus includes all of the methods for producing bispecific antibodies described herein, including for example recombining by half-molecule exchange (also known as “controlled Fab-arm exchange”), as well as recombining at nucleic acid level and/or through co-expression of two half-molecules in the same cells.

The term “full-length” as used herein in the context of an antibody indicates that the antibody is not a fragment but contains all of the domains of the particular isotype normally found for that isotype in nature, e.g., the VH, CH1, CH2, CH3, hinge, VL and CL domains for an IgG1 antibody. A full-length antibody may be engineered. An example of a “full-length” antibody is epcoritamab.

The term “Fc region” as used herein refers to an antibody region consisting of the Fc sequences of the two heavy chains of an immunoglobulin, wherein said Fc sequences comprise at least a hinge region, a CH2 domain, and a CH3 domain.

The term “heterodimeric interaction between the first and second CH3 regions” as used herein refers to the interaction between the first CH3 region and the second CH3 region in a first-CH3/second-CH3 heterodimeric protein.

The term “homodimeric interactions of the first and second CH3 regions” as used herein refers to the interaction between a first CH3 region and another first CH3 region in a first-CH3/first-CH3 homodimeric protein and the interaction between a second CH3 region and another second CH3 region in a second-CH3/second-CH3 homodimeric protein.

The term “isolated antibody” as used herein refers to an antibody which is substantially free of other antibodies having different antigenic specificities. In a preferred embodiment, an isolated bispecific antibody that specifically binds to CD20 and CD3 is in addition substantially free of monospecific antibodies that specifically bind to CD20 or CD3.

The term “CD3” as used herein refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95LI7), a CD3δ (delta) chain (human CD3δ UniProtKB/Swiss-Prot No P04234, or cynomolgus monkey CD3δ UniProtKB/Swiss-Prot No Q95LI8), two CD3ε (epsilon) chains (human CD3ε UniProtKB/Swiss-Prot No P07766, SEQ ID NO: 28); cynomolgus CD3ε UniProtKB/Swiss-Prot No Q95LI5; or rhesus CD3ε UniProtKB/Swiss-Prot No G7NCB9), and a CD3ζ-chain (zeta) chain (human CD3ζ UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

The term “CD3 antibody” or “anti-CD3 antibody” as used herein refers to an antibody which binds specifically to the antigen CD3, in particular human CD3ε (epsilon).

The term “human CD20” or “CD20” refers to human CD20 (UniProtKB/Swiss-Prot No P11836, SEQ ID NO: 29) and includes any variants, isoforms, and species homologs of CD20 which are naturally expressed by cells, including tumor cells, or are expressed on cells transfected with the CD20 gene or cDNA. Species homologs include rhesus monkey CD20 (macaca mulatta; UniProtKB/Swiss-Prot No H9YXP1) and cynomolgus monkey CD20 (Macaca fascicularis; UniProtKB No G7PQ03).

The term “CD20 antibody” or “anti-CD20 antibody” as used herein refers to an antibody which binds specifically to the antigen CD20, in particular to human CD20.

The term “CD3xCD20 antibody”, “anti-CD3xCD20 antibody”, “CD20xCD3 antibody” or “anti-CD20xCD3 antibody” as used herein refers to a bispecific antibody which comprises two different antigen-binding regions, one of which binds specifically to the antigen CD20 and one of which binds specifically to CD3.

The term “DuoBody®-CD3xCD20” as used herein refers to an IgG1 bispecific CD3xCD20 antibody comprising a first heavy and light chain pair as defined in SEQ ID NO: 24 and SEQ ID NO: 25, respectively, and comprising a second heavy and light chain pair as defined in SEQ ID NO: 26 and SEQ ID NO: 27. The first heavy and light chain pair comprises a region which binds to human CD3ε (epsilon), the second heavy and light chain pair comprises a region which binds to human CD20. The first binding region comprises the VH and VL sequences as defined by SEQ ID NOs: 6 and 7, and the second binding region comprises the VH and VL sequences as defined by SEQ ID NOs: 13 and 14. This bispecific antibody can be prepared as described in WO 2016/110576.

Antibodies comprising functional variants of the heavy chain, light chains, VL regions, VH regions, or one or more CDRs of the antibodies of the examples as also provided herein. A functional variant of a heavy chain, a light chain, VL, VH, or CDRs used in the context of an antibody still allows the antibody to retain at least a substantial proportion (at least about 90%, 95% or more) of functional features of the “reference” and/or “parent” antibody, including affinity and/or the specificity/selectivity for particular epitopes of CD20 and/or CD3, Fc inertness and PK parameters such as half-life, Tmax, Cmax. Such functional variants typically retain significant sequence identity to the parent antibody and/or have substantially similar length of heavy and light chains. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J Mol Biol 1970; 48:444-453 algorithm. Exemplary variants include those which differ from heavy and/or light chains, VH and/or VL, and/or CDR regions of the parent antibody sequences mainly by conservative substitutions; e.g., 10, such as 9, 8, 7, 6, 5, 4, 3, 2 or 1 of the substitutions in the variant may be conservative amino acid residue replacements.

Conservative substitutions may be defined by substitutions within the classes of amino acids reflected in the following table:

TABLE 1
Amino acid residue classes for conservative substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Unless otherwise indicated, the following nomenclature is used to describe a mutation: i) substitution of an amino acid in a given position is written as, e.g., K409R which means a substitution of a Lysine in position 409 with an Arginine; and ii) for specific variants the specific three or one letter codes are used, including the codes Xaa and X to indicate any amino acid residue. Thus, the substitution of Lysine with Arginine in position 409 is designated as: K409R, and the substitution of Lysine with any amino acid residue in position 409 is designated as K409X. In case of deletion of Lysine in position 409 it is indicated by K409*.

The term “humanized antibody” as used herein refers to a genetically engineered non-human antibody, which contains human antibody constant domains and non-human variable domains modified to contain a high level of sequence homology to human variable domains. This can be achieved by grafting of the six non-human antibody CDRs, which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e., the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. The VH and VL of the CD3 arm that is used herein in DuoBody®-CD3xCD20 represents a humanized antigen-binding region. Optionally, additional amino acid modifications, which are not necessarily back-mutations, may be applied to obtain a humanized antibody with preferred characteristics, such as affinity and biochemical properties.

The term “human antibody” as used herein refers to antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The VH and VL of the CD20 arm that is used in DuoBody®-CD3xCD20 represents a human antigen-binding region. Human monoclonal antibodies of the invention can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B-lymphocytes or phage display techniques using libraries of human antibody genes. A suitable animal system for preparing hybridomas that secrete human monoclonal antibodies is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known. Human monoclonal antibodies can thus be generated using, e.g., transgenic or transchromosomal mice or rats carrying parts of the human immune system rather than the mouse or rat system. Accordingly, in one embodiment, a human antibody is obtained from a transgenic animal, such as a mouse or a rat, carrying human germline immunoglobulin sequences instead of animal immunoglobulin sequences. In such embodiments, the antibody originates from human germline immunoglobulin sequences introduced in the animal, but the final antibody sequence is the result of said human germline immunoglobulin sequences being further modified by somatic hypermutations and affinity maturation by the endogenous animal antibody machinery (see, e.g., Mendez et al. Nat Genet 1997; 15:146-56). The VH and VL regions of the CD20 arm that is used in DuoBody®-CD3xCD20 represents a human antigen-binding region.

The term “biosimilar” (e.g., of an approved reference product/biological drug) as used herein refers to a biologic product that is similar to the reference product based on data from (a) analytical studies demonstrating that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product). In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. A biosimilar can be, e.g., a presently known antibody having the same primary amino acid sequence as a marketed antibody, but may be made in different cell types or by different production, purification, or formulation methods.

The term “reducing conditions” or “reducing environment” as used herein refers to a condition or an environment in which a substrate, here a cysteine residue in the hinge region of an antibody, is more likely to become reduced than oxidized.

The term “recombinant host cell” (or simply “host cell”) as used herein is intended to refer to a cell into which an expression vector has been introduced, e.g., an expression vector encoding an antibody described herein. Recombinant host cells include, for example, transfectomas, such as CHO, CHO-S, HEK, HEK293, HEK-293F, Expi293F, PER.C6 or NS0 cells, and lymphocytic cells.

The term “diffuse large B-cell lymphoma” or “DLBCL” as used herein refers to a neoplasm of the germinal center B lymphocytes with a diffuse growth pattern and a high-intermediate proliferation index. DLBCL represents approximately 30% of all lymphomas. Subtypes of DLBCL seem to have different outlooks (prognoses) and responses to treatment. DLBCL can affect any age group but occurs mostly in older people (the average age is mid-60s). “Double hit” and “triple hit” DLBCL refers to DLBCL with MYC and BCL2 and/or BCL6 translocations, falling under the category of high-grade B cell lymphoma (HGBCL) with MYC and BCL2 and/or BCL6 translocations, in accordance with the WHO 2016 classification (Swerdlow S H, Campo E, Harris N L, et al. WHO Classification of Tumours of Haematopoietic and Lymphoid Tissues (Revised ed. 4th). Lyon, France: IARC Press (2017), the contents of which are herein incorporated by reference). Follicular lymphoma grade 3B is also often considered to be equivalent to DLBCL and thus treated as such.

The term “R-CHP” as used herein refers to a drug combination containing rituximab, cyclophosphamide, doxorubicin, and prednisone. The term “R-CHP” is also intended to encompass regimens in which the rituximab component is replaced with a biosimilar thereof, and/or branded or generic versions (generic equivalents) of cyclophosphamide, doxorubicin, and/or prednisone, as well as pharmaceutically acceptable salts, isomers, racemates, solvates, complexes and hydrates, anhydrate forms thereof, and any polymorphic or amorphous forms thereof or combinations thereof, are used in the methods described herein.

The term “polatuzumab vedotin” (CAS Number: 1313206-42-6; DrugBank accession number DB12240; Kyoto Encyclopedia of Genes and Genomes (KEGG) entry: D10761) as used herein refers to a human-Mus musculus monoclonal MCDS4409A heavy chain), disulfide with human-Mus musculus monoclonal MCDS4409A chain, dimer, thioether with maleimidocaproyl-valine-citrulline-p-aminobenzyloxycarbonyl monomethylauristatin E. Polatuzumab vedotin is commercially available as Polivy®. In certain embodiments of the methods described herein, polatuzumab vedotin can be replaced with a biosimilar thereof. Accordingly, it will be understood that the term “polatuzumab vedotin” is intended to encompass biosimilars of rituximab. Also encompassed by the term “polatuzumab vedotin” are antibodies which have CDRs, variable regions, or heavy and light chains of polatuzumab vedotin. The biosimilar may be administered according to a standard of care dosage, or at a dose equivalent to the standard of care dosage specified for polatuzumab vedotin.

The term “rituximab” (CAS Number: 174722-31-7; DrugBank—DB00073; Kyoto Encyclopedia of Genes and Genomes (KEGG) entry D02994) as used herein refers to a genetically engineered chimeric human gamma 1 murine constant domain containing monoclonal antibody against human CD20. The chimeric antibody contains human gamma 1 constant domains and is referred to as “C2B8” in U.S. Pat. No. 5,736,137 (the entire content of which is herein incorporated by reference). Rituximab is commercially available, for example, as Rituxan®, MabThera®, or Zytux®. In certain embodiments of the methods described herein, rituximab can be replaced with a biosimilar thereof. Accordingly, it will be understood that the term “rituximab” is intended to encompass biosimilars of rituximab. Also encompassed by the term “rituximab” are antibodies which have CDRs, variable regions, or heavy and light chains of rituximab. Non-limiting examples of biosimilars of rituximab include Truxima® (rituximab-abbs), Ruxience® (rituximab-pvvr), and Rixathon®. The biosimilar may be administered according to a standard of care dosage, or at a dose equivalent to the standard of care dosage specified for rituximab.

The term “cyclophosphamide” as used herein refers to a nitrogen mustard alkylating agent with the chemical name 2H-1,3,2-Oxazaphosphorin-2-amine, N,N-bis(2-chloroethyl)tetrahydro-, 2-oxide (CAS No. 50-18-0) and has the chemical formula C₇H₁₅Cl₂N₂O₂P. It is marketed under trade names such as Endoxan®, Cytoxan®, Neosar®, Procytox®, and Revimmune®. The term “cyclophosphamide” is also intended to encompass branded and generic versions (generic equivalents) of cyclophosphamide, as well as pharmaceutically acceptable salts, isomers, racemates, solvates, complexes and hydrates, anhydrate forms thereof, and any polymorphic or amorphous forms thereof or combinations thereof.

The term “doxorubicin” as used herein refers to an anthracycline antibiotic, closely related to the natural product daunomycin, and like all anthracyclines, it works by intercalating DNA. Doxorubicin is marketed under trade names such as Adriamycin PFS®, Adriamycin RDF®, or Rubex®. Typically, the drug is administered intravenously, in the form of hydrochloride salt (e.g., as doxorubicin hydrochloride). Doxorubicin hydrochloride has the chemical name 5,12-Naphthacenedione, 10-[(3-amino-2,3,6-trideoxy-α-L-lyxo-hexopyranosyl)oxy]-7,8,9,10-tetrahydro-6,8,11-trihydroxy-8-(hydroxyacetyl)-1-methoxy-, hydrochloride, (8S,10S)-(CAS No. 25316-40-9), and has the chemical formula C₂₇H₂₉NO₁₁·HCl. The term “doxorubicin” is also intended to encompass branded and generic versions (generic equivalents) of doxorubicin, as well as pharmaceutically acceptable salts, isomers, racemates, solvates, complexes and hydrates, anhydrate forms thereof, and any polymorphic or amorphous forms thereof or combinations thereof.

“Prednisone” is a synthetic glucocorticoid with anti-inflammatory and immunosuppressive properties. It is a prodrug that is metabolized in the liver to prednisolone, the active form of the drug. Prednisone is marketed under trade names such as Deltasone®, Liquid Pred®, Rayos®, and Orasone®, among others. Prednisone has the chemical name 17,21-dihydroxypregna-1,4-diene-3,11,20-trione (CAS No. 53-03-2). The term “prednisone” is also intended to encompass branded and generic versions (generic equivalents) of prednisone, as well as pharmaceutically acceptable salts, isomers, racemates, solvates, complexes and hydrates, anhydrate forms thereof, and any polymorphic or amorphous forms thereof or combinations thereof.

The term “treatment” refers to the administration of an effective amount of a therapeutically active antibody described herein for the purpose of easing, ameliorating, arresting or eradicating (curing) symptoms or disease states such as DLBCL. Treatment may result in a complete response (CR), partial response (PR), or stable disease (SD), for example, as defined by Lugano criteria and/or LYRIC.

The term “administering” or “administration” as used herein refers to the physical introduction of a composition (or formulation) comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a therapeutic agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. In the methods described herein, the bispecific antibody (e.g., epcoritamab) is administered subcutaneously.

The term “effective amount” or “therapeutically effective amount” refers to an amount effective, at dosages and for periods of time necessary, to achieve a desired therapeutic result. For example, dosages as defined herein for the bispecific antibody (e.g., epcoritamab), i.e., 24 mg or 48 mg, administered subcutaneously can be defined as such an “effective amount” or “therapeutically effective amount”. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects. In some embodiments, patients treated with the methods described herein will show an improvement in ECOG performance status. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or disorder (e.g., cytokine release syndrome) or of suffering a recurrence of disease, inhibits the development or recurrence of the disease.

The term “inhibits growth” of a tumor as used herein includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or 100%.

The term “subject” as used herein refers to a human patient, for example, a human patient with DLBCL. The terms “subject” and “patient” are used interchangeably herein.

The term “buffer” as used herein denotes a pharmaceutically acceptable buffer. The term “buffer” encompasses those agents which maintain the pH value of a solution, e.g., in an acceptable range and includes, but is not limited to, acetate, histidine, TRIS® (tris (hydroxymethyl) aminomethane), citrate, succinate, glycolate and the like. Generally, the “buffer” as used herein has a pKa and buffering capacity suitable for the pH range of about 5 to about 6, preferably of about 5.5.

“Disease progression” or “PD” as used herein refers to a situation in which one or more indices of DLBCL show that the disease is advancing despite treatment. In one embodiment, disease progression is defined based on the Lugano Response Criteria for Malignant Lymphoma (“Lugano criteria”) and/or Lymphoma Response to Immunomodulatory Therapy Criteria (LYRIC). Details regarding the Lugano criteria/classification system, including definitions for complete response (CR), partial response (PR), no response/stable disease (NR/SD), and progressive disease (PD) are provided in Cheson et al. J Clin Oncol 2014; 32:3059-68, the contents of which are incorporated by reference herein (see, in particular, Table 3 in Cheson et al., 2014).

A “surfactant” as used herein is a compound that is typically used in pharmaceutical formulations to prevent drug adsorption to surfaces and or aggregation. Furthermore, surfactants lower the surface tension (or interfacial tension) between two liquids or between a liquid and a solid. For example, an exemplary surfactant can significantly lower the surface tension when present at very low concentrations (e.g., 5% w/v or less, such as 3% w/v or less, such as 1% w/v or less such as 0.4% w/v or less, such as below 0.1% w/v or less, such as 0.04% w/v). Surfactants are amphiphilic, which means they are usually composed of both hydrophilic and hydrophobic or lipophilic groups, thus being capable of forming micelles or similar self-assembled structures in aqueous solutions. Known surfactants for pharmaceutical use include glycerol monooleate, benzethonium chloride, sodium docusate, phospholipids, polyethylene alkyl ethers, sodium lauryl sulfate and tricaprylin (anionic surfactants); benzalkonium chloride, citrimide, cetylpyridinium chloride and phospholipids (cationic surfactants); and alpha tocopherol, glycerol monooleate, myristyl alcohol, phospholipids, poloxamers, polyoxyethylene alkyl ethers, polyoxyethylene castor oil derivatives, polyoxyethylene sorbintan fatty acid esters, polyoxyethylene sterarates, polyoxyl hydroxystearate, polyoxylglycerides, polysorbates such as polysorbate 20 or polysorbate 80, propylene glycol dilaurate, propylene glycol monolaurate, sorbitan esters sucrose palmitate, sucrose stearate, tricaprylin and TPGS (Nonionic and zwitterionic surfactants).

A “diluent” as used herein is one which is pharmaceutically acceptable (safe and non-toxic for administration to a human) and is useful for the preparation of dilutions of the pharmaceutical composition or pharmaceutical formulation (the terms “composition” and “formulation” are used interchangeably herein). Preferably, such dilutions of the composition dilute only the antibody concentration but not the buffer and stabilizer. Accordingly, in one embodiment, the diluent contains the same concentrations of the buffer and stabilizer as is present in the pharmaceutical composition of the invention. Further exemplary diluents include sterile water, bacteriostatic water for injection (BWFI), a pH buffered solution which is preferably an acetate buffer, sterile saline solution such as water for injection, Ringer's solution or dextrose solution. In one embodiment the diluent comprises or consists essentially of acetate buffer and sorbitol.

As used herein, the term “about” refers to a value that is no more than 10% above and no more than 10% below a specified value.

### DLBCL Treatment Regimens

Provided herein are methods of treating DLBCL in a human subject using a bispecific antibody which binds to CD3 and CD20 (“anti-CD3xCD20 antibody”), e.g., an isolated anti-CD3xCD20 antibody such as epcoritamab which binds to human CD3 and human CD20, in combination with polatuzumab vedotin and R-CHP (i.e., rituximab, cyclophosphamide, doxorubicin, and prednisone or an equivalent thereof, e.g. prednisolone). The methods are useful for treating, e.g. DLBCL with histologically confirmed CD20+ disease, such as previously untreated, DLBCL; e.g. DLBCL in which the subject has an International Prognostic Index (IPI) score of 2-5, such as a score of 2, 3, 4 or 5. It is understood that the methods of treating DLBCL (e.g., newly-diagnosed, previously untreated, (IPI 2-5) DLBCL) with a bispecific antibody which binds to both CD3 and CD20 described herein also encompass corresponding uses of the bispecific antibody for treating DLBCL (e.g., newly-diagnosed, previously untreated, (IPI 2-5) DLBCL) in a human subject. In some embodiments, the subject receiving the therapy according to the invention has not received prior therapy for DLBCL or follicular lymphoma Grade 3B. Accordingly, in one aspect, provided herein is a method of treating DLBCL in a human subject, the method comprising administering a bispecific antibody and an effective amount of polatuzumab vedotin, rituximab, cyclophosphamide, doxorubicin, and prednisone or an equivalent thereof, e.g. prednisolone, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose of 24 mg or 48 mg, and wherein polatuzumab vedotin, rituximab, cyclophosphamide, doxorubicin, prednisone or an equivalent thereof, e.g. prednisolone, and the bispecific antibody are administered in 21-day cycles.

In some embodiments, the bispecific antibody is a full-length antibody. In some embodiments, the bispecific antibody is an antibody with an inert Fc region. In some embodiments, the bispecific antibody is a full-length antibody with an inert Fc region.

In some embodiments, the bispecific antibody is administered at a dose of (or a dose of about) 24 mg. In some embodiments, the bispecific antibody is administered at a dose of (or a dose of about) 48 mg.

With regard to the dose of (or dose of about) 24 mg or 48 mg of the bispecific antibody that is to be administered, or any other specified dose, it is understood that this amount refers to the amount of a bispecific antibody representing a full-length antibody, such as epcoritamab as defined in the Examples section. Hence, one may refer to administering a dose of a bispecific antibody of 24 mg as administering a dose of a bispecific antibody described herein, wherein the dose corresponds to a dose of 24 mg of epcoritamab. One of ordinary skill in the art can readily determine the amount of antibody to be administered when, for example, the antibody used differs substantially in molecular weight from the molecular weight of a full-length antibody such as epcoritamab. For instance, the amount of antibody can be calculated by dividing the molecular weight of the antibody by the weight of a full-length antibody such as epcoritamab and multiplying the outcome thereof with the specified dose as described herein. As long as the bispecific antibody (e.g., a functional variant of DuoBody® CD3xCD20) has highly similar features as DuoBody® CD3xCD20, with regard to plasma half-life, Fc inertness, and/or binding characteristics for CD3 and CD20, i.e., with regard to CDRs and epitope binding features, such antibodies are suitable for use in the methods provided herein at a dose described for a full-length antibody such as epcoritamab.

In some embodiments, the dose of bispecific antibody is administered once every week (weekly administration) in 21-day cycles. In one embodiment, the weekly dose of 24 mg or 48 mg is administered for three and one-third 21-day cycles (i.e., 10 times; on day 15 of cycle 1, and days 1, 8, and 15 of cycles 2-4). In one embodiment, the weekly dose of 24 mg is administered for three and one-third 21-day cycles (i.e., 10 times; on day 15 of cycle 1, and days 1, 8, and 15 of cycles 2-4). In one embodiment, the weekly dose of 48 mg is administered for three and one-third 21-day cycles (i.e., 10 times; on day 15 of cycle 1, and days 1, 8, and 15 of cycle 2). In some embodiments, after the weekly administration, one may reduce the interval of administration to once every three weeks. In one embodiment, the administration once every three weeks is performed for at least four 21-day cycles, such as four 21-day cycles (i.e., at least four times, such as four times). In one embodiment, the administration once every three weeks is performed for four 21-day cycles (i.e., four times).

In one embodiment, the weekly dose of the bispecific antibody is administered in 21-day cycles on cycles 5-8 (which may include priming and intermediate doses, as described below), the dose once every three weeks of the bispecific antibody is administered in 21-day cycles on cycles 5-8.

It is understood that the doses referred to herein may also be referred to as a full or a flat dose in the scenarios above wherein, e.g., the weekly dose and/or dose once every three weeks is administered at the same level. Accordingly, when a dose of 48 mg is selected, preferably, at each weekly administration and at each administration once every three weeks, the same dose of 48 mg is administered. Prior to administering the dose, a priming or a priming and subsequent intermediate (second priming) dose may be administered. This may be advantageous as it may help mitigate cytokine release syndrome (CRS) risk and severity, a side-effect that can occur during treatment with the bispecific anti-CD3xCD20 antibody described herein. Such priming, or priming and intermediate doses, are at a lower dose as compared with the flat or full dose.

Accordingly, in some embodiments, prior to administering the weekly dose of 24 mg or 48 mg, a priming dose of the bispecific antibody may be administered. In one embodiment, the priming dose is administered two weeks prior to administering the first weekly dose of 24 mg or 48 mg in cycle 1. In one embodiment, the priming dose is 0.16 mg (or about 0.16 mg) of the full-length bispecific antibody. In one embodiment, the priming dose of 0.16 mg is administered two weeks prior to administering the first weekly dose of 24 mg in cycle 1. In one embodiment, the priming dose of 0.16 mg is administered two weeks prior to administering the first weekly dose of 48 mg in cycle 1.

In some embodiments, after administering the priming dose and prior to administering the weekly dose of 24 mg or 48 mg, an intermediate dose of said bispecific antibody is administered. In one embodiment, the priming dose is administered one week before the intermediate dose (i.e., on day 1 of cycle 1), and the intermediate dose is administered one week before the first weekly dose of 24 mg or 48 mg (i.e., on day 8 of cycle 1). Thus, the priming dose is administered on day 1 and the intermediate dose is administered on day 8 before the first weekly dose of 24 mg or 48 mg on day 15 of cycle 1. In one embodiment, the intermediate dose is 800 μg (0.8 mg) or about 800 μg (0.8 mg) of the full-length bispecific antibody. In one embodiment, the priming dose of 0.16 mg is administered one week before the intermediate dose (i.e., on day 1 of cycle 1) of 0.8 mg, and the intermediate dose is administered one week before the first weekly dose of 24 mg (i.e., on day 8 of cycle 1). In one embodiment, the priming dose of 0.16 mg is administered one week before the intermediate dose (i.e., on day 1 of cycle 1) of 0.8 mg, and the intermediate dose is administered one week before the first weekly dose of 48 mg (i.e., on day 8 of cycle 1).

The methods described herein involve treating human subjects who have DLBCL with a bispecific antibody which binds to CD3 and CD20 in combination with polatuzumab vedotin rituximab, cyclophosphamide, doxorubicin and prednisone or an equivalent thereof, e.g. prednisolone.

In some embodiments, rituximab, cyclophosphamide, doxorubicin, and prednisone or the equivalent thereof, e.g. prednisolone are administered at standard-of-care dosages for R-CHP, e.g., as supported by clinical studies, according to local guidelines, and/or according to relevant local labels.

For example, in some embodiments, polatuzumab vedotin is administered according to relevant local product labels or summary of product characteristics (see, e.g., POLIVY® (polatuzumab vedotin-piiq) prescribing information, available at https://www.accessdata.fda.gov/drugsatfda_docs/label/2019/761121s0001bl.pdf. In some embodiments, a biosimilar of polatuzumab vedotin is used in place of polatuzumab vedotin in the methods described herein.

In some embodiments, rituximab is administered according to relevant local product labels or summary of product characteristics (see, e.g., RITUXAN® (rituximab) prescribing information, available at www.accessdata.fda.gov/drugsatfda_docs/label/2013/103705s54141bl.pdf). In some embodiments, a biosimilar of rituximab is used in place of rituximab in the methods described herein.

In some embodiments, cyclophosphamide is administered according to relevant local product labels or summary of product characteristics (see, e.g., CYCLOPHOSPHAMIDE injection prescribing information, available at www.accessdata.fda.gov/drugsatfda_docs/label/2013/012141s090,012142s1121bl.pdf).

In some embodiments, doxorubicin is administered according to relevant local product labels or summary of product characteristics (see, e.g., ADRIAMYCIN (DOXOrubicin HCl) for Injection (lyophilized) and ADRIAMYCIN (DOXOrubicin HCL) Injection (0.9% sodium chloride and water) prescribing information, available at www.accessdata.fda.gov/drugsatfda_docs/label/2012/062921s0221bl.pdf; Doxorubicin

Hydrochloride for Injection and Doxorubicin Hydrochloride Injection prescribing information available at www.accessdata.fda.gov/drugsatfda_docs/label/2010/050467s0701bl.pdf).

In some embodiments, prednisolone is administered in place of prednisone in the R-CHP regimen.

In one embodiment polatuzumab vedotin is administered according to local guidelines and local label. In some embodiments, polatuzumab vedotin is administered at a dose of (or a dose of about) 1.8 mg/kg. In some embodiments, polatuzumab vedotin is administered intravenously.

In one embodiment, polatuzumab vedotin is administered once every three weeks. In some embodiments, polatuzumab vedotin is administered once every three weeks (Q3W) in 21-day cycles. In some embodiments, administration of polatuzumab vedotin once every three weeks is performed for six 21-day cycles (i.e., six times). In preferred embodiments, polatuzumab vedotin is administered intravenously once every three weeks for six 21-day cycles (i.e., six times) at a dose of 1.8 mg/kg.

In one embodiment, rituximab is administered according to local guidelines and local labels. In some embodiments, rituximab is administered at a dose of (or a dose of about) 375 mg/m². In some embodiments, rituximab is administered intravenously.

In one embodiment, rituximab is administered once every three weeks. In some embodiments, rituximab is administered once every three weeks (Q3W) in 21-day cycles. In some embodiments, administration of rituximab once every three weeks is performed for six 21-day cycles (i.e., six times). In preferred embodiments, rituximab is administered intravenously once every three weeks for six 21-day cycles (i.e., six times) at a dose of 375 mg/m².

In some embodiments, cyclophosphamide is administered according to local guidelines and local labels. In some embodiments, cyclophosphamide is administered at a dose of (or a dose of about) 750 mg/m². In some embodiments, cyclophosphamide is administered intravenously. In some embodiments, cyclophosphamide is administered once every three weeks. In some embodiments, rituximab is administered once every three weeks (Q3W) in 21-day cycles. In some embodiment, administration of cyclophosphamide once every three weeks is performed for six 21-day cycles (i.e., six times). In a preferred embodiment, cyclophosphamide is administered intravenously once every three weeks for six 21-day cycles (i.e., six times) at a dose of 750 mg/m².

In some embodiments, doxorubicin is administered according to local guidelines and local labels. In some embodiments, doxorubicin is administered at a dose of (or a dose of about) 50 mg/m². In some embodiments, doxorubicin is administered intravenously. In some embodiments, doxorubicin is administered once every three weeks. In some embodiments, doxorubicin is administered once every three weeks (Q3W) in 21-day cycles. In some embodiments, administration of doxorubicin once every three weeks is performed for six 21-day cycles (i.e., six times). In a preferred embodiment, doxorubicin is administered intravenously once every three weeks for six 21-day cycles (i.e., six times) at a dose of 50 mg/m².

In a preferred embodiment, polatuzumab vedotin is preferably administered intravenously at a dose of 1.8 mg/kg, rituximab is preferably administered at a dose of 375 mg/m², cyclophosphamide is preferably administered at a dose of 750 mg/m², doxorubicin is preferably administered at a dose of 50 mg/m², wherein rituximab, cyclophosphamide and doxorubicin is preferably administered intravenously once every three weeks for six 21-day cycles.

In some embodiments, prednisone or an equivalent thereof, e.g. prednisolone, is administered according to local guidelines and local labels. In some embodiments, prednisone or an equivalent thereof, e.g. prednisolone, is administered at a dose of (or a dose of about) 100 mg. In some embodiment, prednisone or an equivalent thereof, e.g. prednisolone, is administered intravenously and/or orally. In some embodiments, prednisone or an equivalent thereof, e.g. prednisolone is administered intravenously. In some embodiments, prednisone or an equivalent thereof, e.g. prednisolone, is administered orally.

In one embodiment, prednisone or an equivalent thereof, e.g. prednisolone, is administered once a day for five consecutive days (i.e., days 1-5) in 21-day cycles. In one embodiment, prednisone or an equivalent thereof, e.g. prednisolone, is administered for six 21-day cycles (e.g., on days 1-5 of cycles 1-6 of the 21-day cycles). In one embodiment, prednisone or an equivalent thereof, e.g. prednisolone, is administered once a day on days 1-5 in 21-day cycles. In some embodiments, prednisone or an equivalent thereof, e.g. prednisolone, is administered once a day for five consecutive days (i.e., days 1-5) for six 21-day cycles (e.g., on days 1-5 of cycles 1-6 of the 21-day cycles). In a preferred embodiment, prednisone or an equivalent thereof, e.g. prednisolone, is administered intravenously once a day for five consecutive days (i.e., days 1-5) for six 21-day cycles (e.g., on days 1-5 of cycles 1-6 of the 21-day cycles) at a dose of 100 mg/day. In another preferred embodiment, prednisone or an equivalent thereof, e.g. prednisolone, is administered orally once a day for five consecutive days (i.e., days 1-5) for six 21-day cycles (e.g., on days 1-5 of cycles 1-6 of the 21-day cycles) at a dose of 100 mg/day. In another preferred embodiment, prednisone or an equivalent thereof, e.g. prednisolone, is administered intravenously and/or orally once a day for five consecutive days (i.e., days 1-5) for six 21-day cycles (e.g., on days 1-5 of cycles 1-6 of the 21-day cycles) at a dose of 100 mg/day.

In one embodiment, polatuzumab vedotin (e.g. intravenous), rituximab (e.g., intravenous), cyclophosphamide (e.g., intravenous), doxorubicin (e.g., intravenous), prednisone or an equivalent thereof, e.g. prednisolone (e.g., intravenous or oral), and the bispecific antibody (e.g., subcutaneous) are administered in 21-day cycles, wherein:
(a) the bispecific antibody is administered as follows:
   (i) in cycle 1, a priming dose of 0.16 mg is administered on day 1, an intermediate dose of 0.8 mg is administered on day 8, and a dose of 24 mg is administered on day 15;
   (ii) in cycle 2-4, a dose of 24 mg is administered on days 1, 8, and 15;
   (iii) in cycles 5-8, a dose of 24 mg is administered on day 1;
(b) polatuzumab vedotin, rituximab, cyclophosphamide and, doxorubicin, are administered on day 1 in cycles 1-6; and
(c) prednisone or an equivalent thereof, e.g. prednisolone, is administered on days 1-5 in cycles 1-6.

In one embodiment, polatuzumab vedotin, rituximab (e.g., intravenous), cyclophosphamide (e.g., intravenous), doxorubicin (e.g., intravenous), prednisone or an equivalent thereof, e.g. prednisolone (e.g., intravenous or oral), and the bispecific antibody (e.g., subcutaneous) are administered in 21-day cycles, wherein:
(a) the bispecific antibody is administered as follows:
   (i) in cycle 1, a priming dose of 0.16 mg is administered on day 1, an intermediate dose of 0.8 mg is administered on day 8, and a dose of 48 mg is administered on day 15;
   (ii) in cycle 2-4, a dose of 48 mg is administered on days 1, 8, and 15;
   (iii) in cycles 5-8, a dose of 48 mg is administered on day 1;
(b) polatuzumab vedotin, rituximab, cyclophosphamide and doxorubicin are administered on day 1 in cycles 1-6; and
(c) prednisone or an equivalent thereof, e.g. prednisolone, is administered on days 1-5 in cycles 1-6.

In the two embodiments above, polatuzumab vedotin is preferably administered at a dose of 1.8 mg/kg, rituximab is preferably administered at a dose of 375 mg/m², cyclophosphamide is preferably administered at a dose of 750 mg/m², doxorubicin is preferably administered at a dose of 50 mg/m² and prednisone or an equivalent thereof, e.g. prednisolone, is preferably administered at a dose of 100 mg/day.

In one embodiment, polatuzumab vedotin (e.g. intravenous), rituximab (e.g., intravenous), cyclophosphamide (e.g., intravenous), doxorubicin (e.g., intravenous), prednisone or an equivalent thereof, e.g. prednisolone, (e.g., intravenous or oral), and the bispecific antibody epcoritamab (e.g., subcutaneous) are administered in 21-day cycles, wherein:
(a) the bispecific antibody epcoritamab is administered as follows:
   (i) in cycle 1, a priming dose of 0.16 mg is administered on day 1, an intermediate dose of 0.8 mg is administered on day 8, and a dose of 24 mg is administered on day 15;
   (ii) in cycle 2-4, a dose of 24 mg is administered on days 1, 8, and 15;
   (iii) in cycles 5-8, a dose of 24 mg is administered on day 1;
(b) polatuzumab vedotin, rituximab, cyclophosphamide and doxorubicin are administered on day 1 in cycles 1-6; and
(c) prednisone or an equivalent thereof, e.g. prednisolone, is administered on days 1-5 in cycles 1-6.

In one embodiment, polatuzumab vedotin (e.g. intravenous), rituximab (e.g., intravenous), cyclophosphamide (e.g., intravenous), doxorubicin (e.g., intravenous), prednisone or an equivalent thereof, e.g. prednisolone, (e.g., intravenous or oral), and the bispecific antibody epcoritamab (e.g., subcutaneous) are administered in 21-day cycles, wherein:
(a) the bispecific antibody epcoritamab is administered as follows:
   (i) in cycle 1, a priming dose of 0.16 mg is administered on day 1, an intermediate dose of 0.8 mg is administered on day 8, and a dose of 48 mg is administered on day 15;
   (ii) in cycle 2-4, a dose of 48 mg is administered on days 1, 8, and 15;
   (iii) in cycles 5-8, a dose of 48 mg is administered on day 1;
(b) polatuzumab vedotin, rituximab, cyclophosphamide and doxorubicin are administered on day 1 in cycles 1-6; and
(c) prednisone or an equivalent thereof, e.g. prednisolone, is administered on days 1-5 in cycles 1-6.

In the two embodiments above, polatuzumab vedotin is preferably administered at a dose of 1.8 mg/kg, rituximab is preferably administered at a dose of 375 mg/m², cyclophosphamide is preferably administered at a dose of 750 mg/m², doxorubicin is preferably administered at a dose of 50 mg/m² and prednisone or an equivalent thereof, e.g. prednisolone, is preferably administered at a dose of 100 mg/day.

In one embodiment dosing of the bispecific antibody, polatuzumab and R-CHP in 21-days cycles is as follows:
Bispecific antibody (subcutaneous):
Cycle 1, day 1: Priming dose (0.16 mg)
Cycle 1, day 8: Intermediate dose (0.8 mg)
Cycle 1, day 15: Full dose (24 or 48 mg)
Cycles 2-4, days 1, 8 and 15: Full dose (24 or 48 mg)
Cycles 5-8, day 1: Full dose (24 or 48 mg) every three weeks
Polatuzumab (intravenous):
Cycles 1-6, day 1: 1.8 mg/kg
R-CHP:
Rituximab: Cycles 1-6, day 1: 375 mg/m² (intravenous)
Cyclophosphamide: Cycles 1-6, day 1: 750 mg/m² (intravenous)
Doxorubicin: Cycles 1-6, day 1: 50 mg/m² (intravenous)
Prednisone or equivalent thereof: Cycles 1-6, days 1-6: 100 mg (oral)

In one embodiment, the subject undergoing the treatment with the methods described herein is an adult male or female, at least 18 years old.

In one embodiment, the subject undergoing the treatment with the methods described herein has documented DLBCL (de novo or histologically transformed from follicular lymphoma or nodal marginal zone lymphoma) with histologically confirmed CD20+ disease, inclusive of the following according to WHO 2016 classification. Accordingly, in one embodiment, the subject has DLBCL, NOS (not otherwise specified). In some embodiments, the subject has “double hit” or “triple hit” DLBCL, which are classified in WHO 2016 as HGBCL, with MYC and BCL2 and/or BCL6 translocations. In some embodiments, the subject has follicular lymphoma Grade 3B.

In one embodiment, the subject with DLBCL has an International Prognostic Index (IPI) score 2-5, such as an IPI score of 2, 3, 4, or 5. IPI risk factors include (1) Ann Arbor Stage III or IV, (2) age>60 years, (3) Lactate dehydrogenase level elevated, (4) ECOG performance score≥2, and (5) more than 1 extranodal site.

In one embodiment, the subject with DLBCL has not received prior therapy for DLBCL or follicular lymphoma Grade 3B.

In a further embodiment, the subject has not received prior treatment with a bispecific antibody targeting CD3 and CD20.

In one embodiment, the subject has an Eastern Cooperative Oncology Group (ECOG) performance status (ECOG PS) of 0-2, such as of 0, 1, or 2. Information regarding ECOG PS scores can be found in, e.g., Oken et al, Am J Clin Oncol 1982 December; 5(6):649-55).

In one embodiment, the subject has one or more measurable disease sites as defined as a positron emission tomography/computed tomography (PET/CT) scan demonstrating PET-positive lesion(s) and at least 1 measurable nodal lesion (long axis≥1.5 cm and short axis>1.0 cm) or ≥1 measurable extra-nodal lesion (long axis≥1.0 cm) on CT scan or MRI.

In some embodiments, the subject has laboratory values meeting the following criteria prior to receiving the first dose the first dose of the bispecific antibody:
Absolute neutrophil count (ANC)≥1.0×109/L (growth factor use is allowed if evidence of bone marrow involvement, but subject must not have received growth factor within 14 days prior to screening labs)
Hemoglobin≥8.0 g/dL (RBC transfusions permitted, but subject must not have received blood transfusions within 7 days prior to screening labs)
Platelet count≥75×109/L, or ≥50×109/L if bone marrow infiltration or splenomegaly (platelet transfusions permitted, but subject must not have received blood transfusions within 7 days prior to screening labs)
Serum aspartate transaminase (AST) or alanine transaminase (ALT) level≤3×ULN
Total bilirubin level≤1.5×ULN or ≤5×ULN for subjects with hepatic involvement of disease or of non-hepatic origin. Subjects with Gilbert's syndrome may have total bilirubin levels>1.5×ULN, but direct bilirubin must be <2×ULN
Estimated Creatinine Clearance (CrCl)≥50 mL/min (as calculated by Cockcroft-Gault Formula, modified as needed for factors such as body weight)
Prothrombin time (PT)/International normalized ratio (INR)/Activated partial thromboplastin time (aPTT)≤1.5×ULN, unless receiving anticoagulation

In Further embodiments, the subject:
Must have diagnosis of DLBCL (de novo or histologically transformed from follicular lymphoma or nodal marginal zone lymphoma) with histologically confirmed CD20+ disease, inclusive of the following according to WHO 2016 classification and documented in pathology report:
Must have DLBCL, not otherwise specified (NOS)
Must have high-grade B cell lymphoma with MYC and BCL-2 and/or BCL-6 translocations per WHO 2016 (“double-hit” or “triple-hit”)
   Note: High-grade B-cell lymphomas NOS or other double-/triple-hit lymphomas (with histologies not consistent with DLBCL) are not eligible
Must have follicular lymphoma Grade 3B
Mut have no prior treatment with a bispecific antibody targeting CD3 and CD20
Must have 1 or more measurable disease sites:
Must have a positron emission tomography/computed tomography (PET/CT) scan demonstrating PET-positive lesion(s) and at least 1 measurable nodal lesion (long axis≥1.5 cm and short axis>1.0 cm) or ≥1 measurable extra-nodal lesion (long axis≥1.0 cm) on CT scan or MRI
Must be eligible to receive and have a need for treatment initiation based on symptoms and/or disease burden as per investigator assessment.
Must have Eastern Cooperative Oncology Group (ECOG) performance status 0-2.
Has no unresolved toxicities from prior anticancer therapy, defined as having not resolved to Common Terminology Criteria for Adverse Events (CTCAE, v 5.0), Grade 1, with the exception of alopecia.
Has no current evidence of primary central nervous system (CNS) tumor or known CNS involvement, including leptomeningeal disease, at screening.
Has no history of severe allergic or anaphylactic reactions to anti-CD20 mAb therapy or known significant allergy or intolerance to any component or excipient of epcoritamab or components of study drug combination agents (e.g., lenalidomide, rituximab, etc.)
Must not have had autologous stem cell transplantation within 3 months prior to screening.
Must not have had chemotherapy, non-investigational, or investigational anti-neoplastic agents (except CD20 mAbs) within 4 weeks or 5 half-lives (whichever is shorter) prior to the first dose of epcoritamab.
Has no clinically significant cardiovascular disease, including:
   Myocardial infarction or stroke within 6 months prior to enrollment,
   OR
   The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
   OR
   Other clinically significant electrocardiogram (ECG) abnormalities within 6 months prior to enrollment unless deemed stable and appropriately treated.
Has no clinically significant liver disease, including hepatitis, current alcohol abuse, or cirrhosis.
Does not have active Hepatitis B Virus (HBV) or Hepatitis C Virus (HCV) infection. Subjects who are positive for hepatitis B core antibody (HBcAb), hepatitis B surface antigen (HBsAg), or hepatitis C antibody must have a negative polymerase chain reaction (PCR) result before enrollment. Those who are PCR positive will be excluded.
Has no known history of Human Immunodeficiency Virus (HIV) infection. Note: HIV testing does not need to be conducted at screening unless it is required per local guidelines or institutional standards.
Has no known active bacterial, viral, fungal, mycobacterial, parasitic, or other infection (excluding fungal infections of the nail beds) requiring intravenous (IV) therapy or IV antibiotics within 2 weeks prior to enrollment.
Has no evidence of significant, uncontrolled concomitant diseases that could affect compliance with the protocol or interpretation of results.
Has no history of other prior malignancies, except for the following:
   Malignancy treated with curative intent and with no known active disease present for ≥3 years before the first dose of study drug and felt to be at low risk for recurrence by the treating physician
   Adequately treated non-melanoma skin cancer or lentigo maligna without evidence of disease
   Adequately treated carcinoma in situ without evidence of disease
   Localized prostate cancer, post-radical prostatectomy with non-rising prostate-specific antigen (PSA) levels<0.1 ng/mL
Has not had radiation therapy to target lesion, or major surgery within 4 weeks of enrollment.
Has no Grade>1 neuropathy.
Must not have active tuberculosis (TB) or history of completed treatment for active TB within the past 12 months.
   Note: Interferon gamma release assay (IGRA) testing does not need to be performed at screening unless active or latent tuberculosis is suspected. For subjects with positive IGRA, active pulmonary tuberculosis must be excluded with clinical evaluation and radiologic imaging. Subjects with positive IGRA and no evidence of active disease may be enrolled after treatment for latent tuberculosis infection (recommendation isoniazid monotherapy for total of 6 months) has been initiated.
Has no evidence of CMV viremia (defined as any positive level above the lower limit of detection) at screening.
Has no current autoimmune disease requiring immunosuppressive therapy except for up to 20 mg prednisone daily (or equivalent).
Has no life-threatening illness, medical condition, or organ system dysfunction that, in the Investigator's opinion, could compromise the subject's safety or put the study outcomes at undue risk.
Has no current seizure disorder requiring therapy.
Has no known active SARS-CoV-2 infection. If a subject has signs/symptoms suggestive of SARS-CoV-2 infection or have recent known exposure to someone with SARS-CoV infection, they should undergo molecular (e.g., PCR) testing or 2 negative antigen test results at least 24 hours apart to rule out SARS-CoV-2 infection.
Subjects who do not meet SARS-CoV-2 infection eligibility criteria must be screen failed and may only rescreen after they meet the following SARS-CoV-2 infection viral clearance criteria:
   At least 10 days since first positive test result have passed in asymptomatic patients or at least 10 days since recovery, defined as resolution of fever without use of antipyretics and improvement in symptoms
Must not have had major surgery within 4 weeks of the first dose of study drug.

In one embodiment, the subject has no current evidence of primary central nervous system (CNS) tumor or known CNS involvement, including leptomeningeal disease, at screening The subject may have no history of severe allergic or anaphylactic reactions to anti-CD20 monoclonal antibody therapy or known significant allergy or intolerance to any component or excipient of epcoritamab or components of study drug combination agents (e.g., lenalidomide, rituximab, etc.)

In one embodiment, the subject must not have had autologous stem cell transplantation within 3 months prior to screening.

In one embodiment, the subject must not have had chemotherapy, non-investigational, or investigational anti-neoplastic agents (except CD20 monoclonal antibodies) within 4 weeks or 5 half-lives (whichever is shorter) prior to the first dose of epcoritamab.

In one embodiment, the subject has no clinically significant cardiovascular disease, including:
Myocardial infarction or stroke within 6 months prior to enrollment,
OR
The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
OR
Other clinically significant electrocardiogram (ECG) abnormalities within 6 months prior to enrollment unless deemed stable and appropriately treated.
Left ventricular ejection fraction (LVEF) must be within institutional normal limits by multi-gated acquisition (MUGA) or transthoracic echocardiography at screening.

In one embodiment, the subject has no history of other prior malignancies, except for the following:
Malignancy treated with curative intent and with no known active disease present for ≥3 years before the first dose of study drug and felt to be at low risk for recurrence by treating physician
Adequately treated non-melanoma skin cancer or lentigo maligna without evidence of disease
Adequately treated carcinoma in situ without evidence of disease;
Localized prostate cancer, post-radical prostatectomy with non-rising prostate-specific antigen (PSA) levels<0.1 ng/mL

In one embodiment, the subject has not had radiation therapy to target lesion, or major surgery within 4 weeks of enrollment.

In one embodiment, the subject has no Grade>1 neuropathy

A human subject receiving a treatment described herein may be a patient having one or more of the inclusion criteria set forth in Example 3, or not having one or more of the exclusion criteria set forth in Example 3.

The methods described herein are advantageous for treating DLBCL, such as with an International Prognostic Index (IPI) score of 2-5 such as 2, 3, 4 or 5. The treatment is maintained continuously using, e.g., the treatment regimens described herein. However, treatment may be terminated when progressive disease develops or unacceptable toxicity occurs.

In one embodiment the method of the invention is for 1^{st}-line treatment of DLBCL.

The response of subjects with DLBCL to treatment using the methods described herein may be assessed according to the Lugano Response Criteria for Malignant Lymphoma (also referred to as “Lugano criteria” herein) and/or Lymphoma Response to Immunomodulatory Therapy Criteria (also referred to as “LYRIC” herein), as described in Example 3. In one embodiment, complete response (CR), partial response (PR), and stable disease (SD) are assessed using the Lugano criteria. In some embodiments, patients showing disease progression, also referred to as progressive disease (PD), according to the Lugano criteria are further evaluated according to LYRIC. Details regarding the Lugano criteria/classification system, including definitions for complete response, partial response, no response/stable disease, and progressive disease are provided in Cheson et al. J Clin Oncol 2014; 32:3059-68 (see, in particular, Table 3 in Cheson et al., 2014). Details regarding LYRIC are provided in Example 3 herein.

In some embodiments, subjects are treated with the methods described herein until they show disease progression (PD), e.g., as defined by Lugano criteria and/or LYRIC. In one embodiment, subjects are treated with the methods described herein until they show disease progression (PD) as defined by both Lugano criteria and LYRIC.

Subjects treated according to the methods described herein preferably experience improvement in at least one sign of DLBCL. In one embodiment, improvement is measured by a reduction in the quantity and/or size of measurable tumor lesions. In some embodiments, lesions can be measured on CT (computed tomography), PET-CT (positron emission tomography-computed tomography), or MRI (magnetic resonance imaging) films. In some embodiments, cytology or histology can be used to evaluate responsiveness to a therapy. In some embodiments, bone marrow aspirate, bone marrow biopsy, tumor biopsy, physical examination and/or laboratory tests (e.g., tumor cells in ascites or pleural fluid) can be used to evaluate response to therapy.

In one embodiment, the subject treated exhibits a complete response (CR), a partial response (PR), or stable disease (SD), as defined by the Lugano criteria and/or LYRIC (see, e.g., Example 3 herein). In some embodiments, the methods described herein produce at least one therapeutic effect chosen from prolonged survival, such as progression-free survival or overall survival, optionally compared to another therapy, such as treatment with R-CHP alone.

In one embodiment, the bispecific antibody used in the methods described herein is administered subcutaneously, and thus is formulated in a pharmaceutical composition such that it is compatible with subcutaneous (s.c.) administration, i.e., having a formulation and/or concentration that allows pharmaceutical acceptable s.c. administration at the doses described herein. In some embodiments, subcutaneous administration is carried out by injection. For example, formulations for DuoBody® CD3xCD20 that are compatible with subcutaneous formulation and can be used in the methods described herein have been described previously (see, e.g., WO2019155008, which is incorporated herein by reference). In some embodiments, the bispecific antibody may be formulated using sodium acetate trihydrate, acetic acid, sodium hydroxide, sorbitol, polysorbate 80, and water for injection, and have a pH of 5.5 or about 5.5. In some embodiments, the bispecific antibody is provided as a 5 mg/mL or 60 mg/mL concentrate. In other embodiments, the desired dose of the bispecific antibody is reconstituted to a volume of about 1 mL for subcutaneous injection.

In one embodiment, a suitable pharmaceutical composition for the bispecific antibody can comprise the bispecific antibody, 20-40 mM acetate, 140-160 mM sorbitol, and a surfactant, such as polysorbate 80, and having a pH of 5.3-5.6. In some embodiments, the pharmaceutical formulation may comprise an antibody concentration in the range of 5-100 mg/mL, e.g., 48 or 60 mg/mL of the bispecific antibody, 30 mM acetate, 150 mM sorbitol, 0.04% w/v polysorbate 80, and have a pH of 5.5. Such a formulation may be diluted with, e.g., the formulation buffer to allow proper dosing and subcutaneous administration.

The volume of the pharmaceutical composition is appropriately selected to allow for subcutaneous administration of the antibody. For example, the volume to be administered is in the range of about 0.3 mL to about 3 mL, such as from 0.3 mL to 3 mL. The volume to be administered can be 0.5 mL, 0.8 mL, 1 mL, 1.2 mL, 1.5 ml, 1.7 mL, 2 mL, or 2.5 mL, or about 0.5 mL, about 0.8 mL, about 1 mL, about 1.2 mL, about 1.5 ml, about 1.7 mL, about 2 mL, or about 2.5 mL. Accordingly, in one embodiment, the volume to be administered is 0.5 mL or about 0.5 mL. In some embodiments, the volume to be administered is 0.8 mL or about 0.8 mL. In some embodiments, the volume to be administered is 1 mL or about 1 mL. In some embodiments, the volume to be administered is 1.2 mL or about 1.2 mL. In some embodiments, the volume to be administered is 1.5 mL or about 1.5 mL. In some embodiments, the volume to be administered is 1.7 mL or about 1.7 mL. In some embodiments, the volume to be administered is 2 mL or about 2 mL. In some embodiments, the volume to be administered is 2.5 mL or about 2.5 mL.

In one embodiment, rituximab is formulated in a pharmaceutical composition comprising pharmaceutically-acceptable excipients for administration (e.g., intravenous administration) in accordance with local standard-of-care practice, e.g., as specified by local guidelines or local product labels. For example, in some embodiments, rituximab is provided as a sterile, clear, colorless, preservative-free liquid concentrate for intravenous administration. In one embodiment, rituximab is supplied at a concentration of 10 mg/mL in either 100 mg/10 mL or 500 mg/50 mL single-use vials. In some embodiments, rituximab is formulated in polysorbate 80 (0.7 mg/mL), sodium citrate dihydrate (7.35 mg/mL), sodium chloride (9 mg/mL), and water, at a pH of 6.5, for injection.

In one embodiment, polatuzumab vedotin, cyclophosphamide, doxorubicin and prednisone are formulated in a pharmaceutical composition comprising pharmaceutically-acceptable excipients for administration (e.g., intravenous administration) in accordance with local standard-of-care practice, e.g., as specified by local guidelines or local product labels, or as directed by the manufacturer. In some embodiments, polatuzumab vedotin, cyclophosphamide, doxorubicin and prednisone are diluted from a stock solution, or reconstituted if in lyophilized form, according to, e.g., instructions in the product label (e.g., with 0.9% saline solution). In some embodiments, prednisone is formulated in a pharmaceutical composition for oral administration.

In one embodiment, the bispecific antibody used in the methods described herein comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences within the amino acid sequence of SEQ ID NO: 6, and the VL region comprises the CDR1, CDR2 and CDR3 sequences within the amino acid sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences within the amino acid sequence of SEQ ID NO: 13, and the VL region comprises the CDR1, CDR2 and CDR3 sequences within the amino acid sequence SEQ ID NO: 14.

CDR1, CDR2 and CDR3 regions can be identified from variable heavy and light chain regions using methods known in the art. The CDR regions from said variable heavy and light chain regions can be annotated according to IMGT (see Lefranc et al., Nucleic Acids Research 1999; 27:209-12, 1999] and Brochet. Nucl Acids Res 2008; 36:W503-8).

In one embodiment, the bispecific antibody comprises:
(i) a first binding arm comprising a first antigen-binding region which binds to human CD3ε (epsilon) and comprises VHCDR1, VHCDR2 and VHCDR3 the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
(ii) a second binding arm comprising a second antigen-binding region which binds to human CD20 and comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.

In one embodiment, the bispecific antibody comprises:
(i) a first binding arm comprising a first antigen-binding region which binds to human CD3ε (epsilon) and comprises a VH region comprising the amino acid sequence of SEQ ID NO: 6, and a VL region comprising the amino acid sequence of SEQ ID NO: 7; and
(ii) a second binding arm comprising a second antigen-binding region which binds to human CD20 and comprises a VH region comprising the amino acid sequence of SEQ ID NO: 13, and a VL region comprising the amino acid sequence of SEQ ID NO: 14.

In one embodiment, the bispecific antibody is a full-length antibody. In some embodiments, the bispecific antibody has an inert Fc region. In some embodiments, the bispecific antibody is a full-length antibody and have an inert Fc region. In some embodiments, the first binding arm for CD3 is derived from a humanized antibody, e.g., from a full-length IgG1,λ (lambda) antibody such as H1L1 described in WO2015001085, which is incorporated herein by reference, and/or the second binding arm for CD20 is derived from a human antibody, e.g., from a full-length IgG1,κ (kappa) antibody such as clone 7D8 as described in WO2004035607, which is incorporated herein by reference. The bispecific antibody may be produced from two half molecule antibodies, wherein each of the two half molecule antibodies comprising, e.g., the respective first and second binding arms set forth in SEQ ID NOs: 24 and 25, and SEQ ID NOs: 26 and 27. The half-antibodies may be produced in CHO cells and the bispecific antibodies generated by, e.g., Fab-arm exchange. In one embodiment, the bispecific antibody is a functional variant of DuoBody® CD3xCD20.

Accordingly, in some embodiments, the bispecific antibody comprises (i) a first binding arm comprising a first antigen-binding region which binds to human CD3ε (epsilon) and comprises a VH region comprising an amino acid sequence which is at least 85%, 90%, 95%, 96%, 97%, 98%, or 99% identical to SEQ ID NO: 6 or a VH region comprising the amino acid sequence of SEQ ID NO: 6, but with 1, 2, or 3 mutations (e.g., amino acid substitutions), and a VL region comprising an amino acid sequence which is at least 85%, 90%, 95%, 96%, 97%, 98%, or 99% identical to SEQ ID NO: 7 or a VL region comprising the amino acid sequence of SEQ ID NO: 7, but with 1, 2, or 3 mutations (e.g., amino acid substitutions); and
(ii) a second binding arm comprising a second antigen-binding region which binds to human CD20 and comprises a VH region comprising an amino acid sequence which is at least 85%, 90%, 95%, 98%, or 99% identical to SEQ ID NO: 13 or a VH region comprising the amino acid sequence of SEQ ID NO: 13, but with 1, 2, or 3 mutations (e.g., amino acid substitutions), and a VL region comprising an amino acid sequence which is at least 85%, 90%, 95%, 98%, or 99% identical to SEQ ID NO: 14 or a VL region comprising the amino acid sequence of SEQ ID NO: 14, but with 1, 2, or 3 mutations (e.g., amino acid substitutions).

In one embodiment, the bispecific antibody comprises:
(i) a first binding arm comprising a first antigen-binding region which binds to human CD3ε (epsilon) and comprises a heavy chain comprising the amino acid sequence of SEQ ID NO: 24, and a light chain comprising the amino acid sequence of SEQ ID NO: 25; and
(ii) a second binding arm comprising a second antigen-binding region which binds to human CD20 and comprises a VH region comprising the amino acid sequence of SEQ ID NO: 26, and a VL region comprising the amino acid sequence of SEQ ID NO: 27.

In some embodiments, the bispecific antibody comprises (i) a first binding arm comprising a first antigen-binding region which binds to human CD3ε (epsilon) and comprises a heavy chain comprising an amino acid sequence which is at least 85%, 90%, 95%, 98%, or 99% identical to SEQ ID NO: 24 or a heavy chain comprising the amino acid sequence of SEQ ID NO: 24, but with 1, 2, or 3 mutations (e.g., amino acid substitutions), and a light chain comprising an amino acid sequence which is at least 85%, 90%, 95%, 98%, or 99% identical to SEQ ID NO: 25 or a light chain region comprising the amino acid sequence of SEQ ID NO: 25, but with 1, 2, or 3 mutations (e.g., amino acid substitutions); and
(ii) a second binding arm comprising a second antigen-binding region which binds to human CD20 and comprises a heavy chain comprising an amino acid sequence which is at least 85%, 90%, 95%, 98%, or 99% identical to SEQ ID NO: 26 or a heavy chain comprising the amino acid sequence of SEQ ID NO: 26, but with 1, 2, or 3 mutations (e.g., amino acid substitutions), and a light chain comprising an amino acid sequence which is at least 85%, 90%, 95%, 98%, or 99% identical to SEQ ID NO: 27 or a light chain region comprising the amino acid sequence of SEQ ID NO: 27, but with 1, 2, or 3 mutations (e.g., amino acid substitutions).

Various constant regions or variants thereof may be used in the bispecific antibody. In one embodiment, the antibody comprises an IgG constant region, such as a human IgG1 constant region, e.g., a human IgG1 constant region as defined in SEQ ID NO: 15, or any other suitable IgG1 allotype. In one embodiment, the bispecific antibody is a full-length antibody with a human IgG1 constant region. In one embodiment, the first binding arm of the bispecific antibody is derived from a humanized antibody, preferably from a full-length IgG1,λ (lambda) antibody. In one embodiment, the first binding arm of the bispecific antibody is derived from a humanized antibody, e.g., from a full-length IgG1,λ (lambda) antibody, and thus comprises a λ light chain constant region. In some embodiments, the first binding arm comprises a λ light chain constant region as defined in SEQ ID NO: 22. In one embodiment, the second binding arm of the bispecific antibody is derived from a human antibody, preferably from a full-length IgG1,κ (kappa) antibody. In one embodiment the second binding arm of the bispecific antibody is derived from a human antibody, preferably from a full-length IgG1,κ (kappa) antibody, and thus may comprise a κ light chain constant region. In some embodiments, the second binding arm comprises a κ light chain constant region as defined in SEQ ID NO: 23. In a preferred embodiment, the first binding arm comprises a λ light chain constant region as defined in SEQ ID NO: 22 and the second binding arm comprises a κ light chain constant region as defined in SEQ ID NO: 23.

It is understood that the constant region portion of the bispecific antibody may comprise modifications that allow for efficient formation/production of bispecific antibodies and/or provide for an inert Fc region. Such modifications are well known in the art.

Different formats of bispecific antibodies are known in the art (reviewed by Kontermann, Drug Discov Today 2015; 20:838-47; MAbs, 2012; 4:182-97). Thus, the bispecific antibody used in the methods and uses described herein are not limited to any particular bispecific format or method of producing it. For example, bispecific antibodies may include, but are not limited to, bispecific antibodies with complementary CH3 domains to force heterodimerization, Knobs-into-Holes molecules (Genentech, WO9850431), CrossMAbs (Roche, WO2011117329), or electrostatically-matched molecules (Amgen, EP1870459 and WO2009089004; Chugai, US201000155133; Oncomed, WO2010129304).

Preferably, the bispecific antibody comprises an Fc-region comprising a first heavy chain with a first Fc sequence comprising a first CH3 region, and a second heavy chain with a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions. Further details on these interactions and how they can be achieved are provided in e.g. WO2011131746 and WO2013060867 (Genmab), which are hereby incorporated by reference. In one embodiment, the bispecific antibody comprises in the first heavy chain (i) the amino acid L in the position corresponding to F405 in the human IgG1 heavy chain constant region of SEQ ID NO: 15, and comprises in the second heavy chain the amino acid R in the position corresponding to K409 in the human IgG1 heavy chain constant region of SEQ ID NO: 15, or vice versa.

Bispecific antibodies may comprise modifications in the Fc region to render the Fc region inert, or non-activating. Thus, in the bispecific antibodies disclosed herein, one or both heavy chains may be modified so that the antibody induces Fc-mediated effector function to a lesser extent relative to the bispecific antibody which does not have the modification. Fc-mediated effector function may be measured by determining Fc-mediated CD69 expression on T cells (i.e. CD69 expression as a result of CD3 antibody-mediated, Fcγ receptor-dependent CD3 crosslinking), by binding to Fcγ receptors, by binding to C1q, or by induction of Fc-mediated cross-linking of FcγRs. In particular, the heavy chain constant region sequence may be modified so that Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g. as described in Example 3 of WO2015001085. Modifications of the heavy and light chain constant region sequences may also result in reduced binding of C1q to said antibody. As compared to an unmodified antibody, the reduction may be by at least 70%, at least 80%, at least 90%, at least 95%, at least 97%, or 100%, and C1q binding may be determined, e.g., by ELISA. Further, the Fc region which may be modified so that the antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay. Examples of amino acid positions that may be modified, e.g., in an IgG1 isotype antibody, include positions L234 and L235. Thus, in one embodiment, the bispecific antibody may comprise a first heavy chain and a second heavy chain, and wherein in both the first heavy chain and the second heavy chain, the amino acid residues at the positions corresponding to positions L234 and L235 in a human IgG1 heavy chain according to Eu numbering are F and E, respectively. In addition, a D265A amino acid substitution can decrease binding to all Fcγ receptors and prevent ADCC (Shields et al., JBC 2001; 276:6591-604). Therefore, the bispecific antibody may comprise a first heavy chain and a second heavy chain, wherein in both the first heavy chain and the second heavy chain, the amino acid residue at the position corresponding to position D265 in a human IgG1 heavy chain according to Eu numbering is A.

In one embodiment, in the first heavy chain and second heavy chain of the bispecific antibody, the amino acids in the positions corresponding to positions L234, L235, and D265 in a human IgG1 heavy chain, are F, E, and A, respectively. An antibody having these amino acids at these positions is an example of an antibody having an inert Fc region, or a non-activating Fc region. In one embodiment, the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively. In one embodiment, the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa. In a preferred embodiment, the bispecific antibody comprises a first heavy chain and a second heavy chain, wherein (i) in both the first and second heavy chains, the amino acids in the positions corresponding to positions L234, L235, and D265 in the human IgG1 heavy chain constant region of SEQ ID NO: 15 are F, E, and A, respectively, and (ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.

With regards to the bispecific antibodies described herein, those which have the combination of three amino acid substitutions L234F, L235E and D265A and in addition the K409R or the F405L mutation, as described above, may be referred to with the suffix “FEAR” or “FEAL”, respectively.

An amino acid sequence of a wild type IgG1 heavy chain constant region may be identified herein as SEQ ID NO: 15. Consistent with the embodiments disclosed above, the bispecific antibody may comprise an IgG1 heavy chain constant region carrying the F405L substitution and may have the amino acid sequence set forth in SEQ ID NO: 17 and/or an IgG1 heavy chain constant region carrying the K409R substitution and may have the amino acid sequence set forth in SEQ ID NO: 18, and have further substitutions that render the Fc region inert or non-activating. Hence, in one embodiment, the bispecific antibody comprises a combination of IgG1 heavy chain constant regions, with the amino acid sequence of one of the IgG1 heavy chain constant regions carrying the L234F, L235E, D265A and F405L substitutions (e.g., as set forth in SEQ ID NO: 19) and the amino acid sequence of the other IgG1 heavy chain constant region carrying the L234F, L235E, D265A and K409R substitutions (e.g., as set forth in SEQ ID NO: 20). Thus, in one embodiment, the bispecific antibody comprises heavy chain constant regions comprising the amino acid sequences of SEQ ID NOs: 19 and 20.

In preferred embodiments, the bispecific antibody used in the methods and uses described herein comprises a first binding arm comprising a heavy chain and a light chain as defined in SEQ ID NOs: 24 and 25, respectively, and a second binding arm comprising a heavy chain and a light chain as defined in SEQ ID NOs: 26 and 27, respectively. Such an antibody is referred to herein as DuoBody® CD3xCD20. Also, variants of such antibodies are contemplated use in the methods and uses as described herein. In some embodiment, the bispecific antibody comprising a heavy chain and a light chain consisting of the amino acid sequences set forth in SEQ ID NOs: 24 and 25, respectively, and a heavy chain and a light chain consisting of the amino acid sequences set forth in SEQ ID NOs: 26 and 27, respectively. In some embodiments, the bispecific antibody is epcoritamab (CAS 2134641-34-0), or a biosimilar thereof.

### Medical Use

Further provided herein is a bispecific antibody for use in a method as disclosed above.

In particular embodiments, the bispecific antibody is for use in a method of treating diffuse large B-cell lymphoma (DLBCL) in a human subject, wherein the bispecific antibody is administered to a subject in combination with an effective amount of (a) polatuzumab vedotin, (b) rituximab, (c) cyclophosphamide, (d) doxorubicin and (e) prednisone or an equivalent thereof, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose of 24 mg or 48 mg, and wherein polatuzumab vedotin, rituximab, cyclophosphamide, doxorubicin, prednisone or an equivalent thereof, and the bispecific antibody are administered in 21-day cycles.

Also provided herein is a bispecific antibody for the manufacture of a medicament for use in a method as disclosed above.

In particular, the bispecific antibody is for the manufacture of a medicament for use in treating diffuse large B-cell lymphoma (DLBCL) in a human subject, wherein the bispecific antibody is administered to a subject in combination with an effective amount of (a) polatuzumab vedotin, (b) rituximab, (c) cyclophosphamide, (d) doxorubicin and (e) prednisone or an equivalent thereof, wherein the bispecific antibody comprises:
(i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
(ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
wherein the bispecific antibody is administered at a dose of 24 mg or 48 mg, and wherein polatuzumab vedotin, rituximab, cyclophosphamide, doxorubicin, prednisone or an equivalent thereof, and the bispecific antibody are administered in 21-day cycles.

### Kits

Also provided herein are kits which include a pharmaceutical composition containing a bispecific antibody which binds to CD3 and CD20 in accordance with the invention, such as DuoBody® CD3xCD20 or epcoritamab, and a pharmaceutically-acceptable carrier, in a therapeutically effective amount adapted for use in the methods described herein. The kits may also include a pharmaceutical composition containing polatuzumab vedotin (e.g. for intravenous administration), rituximab (e.g., for intravenous administration), cyclophosphamide (e.g., for intravenous administration), doxorubicin (e.g., for intravenous administration), and/or prednisone or an equivalent thereof (e.g., for intravenous or oral administration). The kits optionally also can include instructions, e.g., comprising administration schedules, to allow a practitioner (e.g., a physician, nurse, or patient) to administer the composition or compositions contained therein to a patient with DLBCL. The kit also can include a syringe or syringes.

Optionally, the kits include multiple packages of the single-dose pharmaceutical compositions each containing an effective amount of the bispecific antibody for a single administration in accordance with the methods described herein. They may also include multiple packages of single dose pharmaceutical compositions containing a dose of polatuzumab vedotin, rituximab, cyclophosphamide, doxorubicin, and/or prednisone or an equivalent thereof, in accordance with a standard of care regimen. Instruments or devices necessary for administering the pharmaceutical composition(s) also may be included in the kits.

### Further Embodiments

1. A method of treating diffuse large B-cell lymphoma (DLBCL) in a human subject, the method comprising administering to the subject a bispecific antibody, and an effective amount of (a) polatuzumab vedotin, (b) rituximab, (c) cyclophosphamide, (d) doxorubicin and (e) prednisone or an equivalent thereof, wherein the bispecific antibody comprises:
   (i) a first binding arm comprising a first antigen-binding region which binds to human CD3ε (epsilon) and comprises a variable heavy chain (VH) region and a variable light chain (VL) region, wherein the VH region comprises the CDR1, CDR2 and CDR3 sequences that are in the VH region sequence of SEQ ID NO: 6, and the VL region comprises the CDR1, CDR2 and CDR3 sequences that are in the VL region sequence of SEQ ID NO: 7; and
   (ii) a second binding arm comprising a second antigen-binding region which binds to human CD20 and comprises a VH region and a VL region, wherein the VH region comprises the CDR1, CDR2 and CDR3 sequences that are in the VH region sequence of SEQ ID NO: 13, and the VL region comprises the CDR1, CDR2 and CDR3 sequences that are in the VL region sequence of SEQ ID NO: 14;
   wherein the bispecific antibody is administered at a dose of 24 mg or 48 mg, and wherein polatuzumab vedotin, rituximab, cyclophosphamide, doxorubicin, prednisone or an equivalent thereof, and the bispecific antibody are administered in 21-day cycles.
2. The method of embodiment 1, wherein the bispecific antibody is administered at a dose of 24 mg.
3. The method of embodiment 1, wherein the bispecific antibody is administered at a dose of 48 mg.
4. The method of any one of embodiments 1-3, wherein the bispecific antibody is administered once every week (weekly administration).
5. The method of embodiment 4, wherein the weekly administration of 24 mg or 48 mg is performed for three and one-third 21-day cycles.
6. The method of embodiment 4 or 5, wherein after the weekly administration, the bispecific antibody is administered once every three weeks, such as in 21-day cycles, on day 1 of each 21-day cycle.
7. The method of embodiment 6, wherein the administration once every three weeks is performed for at least four 21-day cycles.
8. The method of embodiment 7, wherein the administration once every three weeks is performed for four 21-day cycles.
9. The method of any one of embodiments 4-8, wherein prior to the weekly administration of 24 mg or 48 mg, a priming dose of the bispecific antibody is administered in cycle 1 of the 21-day cycles.
10. The method of embodiment 9, wherein the priming dose is administered two weeks prior to administering the first weekly dose of 24 mg or 48 mg.
11. The method of embodiment 9 or 10, wherein the priming dose is 0.16 mg.
12. The method of any one of embodiments 9-11, wherein after administering the priming dose and prior to administering the first weekly dose of 24 mg or 48 mg, an intermediate dose of the bispecific antibody is administered.
13. The method of embodiment 12, wherein the priming dose is administered on day 1 and the intermediate dose is administered on day 8 before the first weekly dose of 24 mg or 48 mg on day 15 of cycle 1.
14. The method of embodiment 12 or 13, wherein the intermediate dose is 0.8 mg.
15. The method of any one of embodiments 1-14, wherein the bispecific antibody is administered subcutaneously.
16. The method of any one of embodiments 1-15, wherein polatuzumab vedotin is administered once every three weeks.
17. The method of any one of embodiments 1-16, wherein the administration of polatuzumab vedotin once every three weeks is performed for at six 21-day cycles.
18. The method of any one of embodiments 1-17, wherein polatuzumab vedotin is administered at a dose of 1.8 mg/kg.
19. The method of any one of embodiments 1-18, wherein polatuzumab vedotin is administered on day 1 of each 21-day cycle.
20. The method of any one of embodiments 1-18, wherein rituximab is administered once every three weeks.
21. The method of embodiment 20, wherein the administration of rituximab once every three weeks is performed for six 21-day cycles.
22. The method of any one of embodiments 1-21, wherein rituximab is administered at a dose of 375 mg/m².
23. The method of any one of embodiments 1-22, wherein rituximab is administered on day 1 of each 21-day cycle.
24. The method of any one of embodiments 1-23, wherein cyclophosphamide is administered once every three weeks.
25. The method of embodiment 24, wherein the administration of cyclophosphamide once every three weeks is performed for six 21-day cycles.
26. The method of any one of embodiments 1-25, wherein cyclophosphamide is administered at a dose of 750 mg/m².
27. The method of any one of embodiments 1-26, wherein cyclophosphamide is administered on day 1 of each 21-day cycle.
28. The method of any one of embodiments 1-27, wherein doxorubicin is administered once every three weeks.
29. The method of embodiment 28, wherein the administration of doxorubicin once every three weeks is performed for six 21-day cycles.
30. The method of any one of embodiments 1-29, wherein doxorubicin is administered at a dose of 50 mg/m².
31. The method of any one of embodiments 1-30, wherein doxorubicin is administered on day 1 of each 21-day cycle.
32. The method of any one of embodiments 1-31, wherein the equivalent of prednisone is prednisolone.
33. The method of any one of embodiments 1-32, wherein prednisone or prednisolone is administered once a day from day 1 to day 5 of the 21-day cycles.
34. The method of embodiment 33, wherein prednisone or prednisolone is administered for six 21-day cycles.
35. The method of any one of embodiments 1-34, wherein prednisone or prednisolone is administered at a dose of 100 mg/day.
36. The method of any one of embodiments 1-35, wherein prednisone or prednisolone is administered on days 1-5 of each 21-day cycle.
37. The method of any one of embodiments 1-36, wherein polatuzumab vedotin, rituximab, cyclophosphamide, doxorubicin, prednisone or the equivalent thereof, and the bispecific antibody are administered on the same day (e.g., on day 1 of cycles 1-6 or cycles 1-8 of the 21-day cycles).
38. The method of any one of embodiments 1, 2, and 4-37, wherein administration is performed in 21-day cycles, and wherein:
   (a) the bispecific antibody is administered as follows:
      (i) in cycle 1, a priming dose of 0.16 mg is administered on day 1, an intermediate dose of 0.8 mg is administered on day 8, and a dose of 24 mg is administered on day 15;
      (ii) in cycle 2-4, a dose of 24 mg is administered on days 1, 8, and 15;
      (iii) in cycles 5-8, a dose of 24 mg is administered on day 1;
   (b) polatuzumab vedotin, rituximab, cyclophosphamide and doxorubicin are administered on day 1 in cycles 1-6; and
   (c) prednisone or the equivalent thereof is administered on days 1-5 in cycles 1-6.
39. The method of any one of embodiments 1 and 3-37, wherein administration is performed in 21-day cycles, and wherein:
   (a) the bispecific antibody is administered as follows:
      (i) in cycle 1, a priming dose of 0.16 mg is administered on day 1, an intermediate dose of 0.8 mg is administered on day 8, and a dose of 48 mg is administered on day 15;
      (ii) in cycle 2-4, a dose of 48 mg is administered on days 1, 8, and 15;
      (iii) in cycles 5-8, a dose of 48 mg is administered on day 1;
(b) polatuzumab vedotin, rituximab, cyclophosphamide and doxorubicin are administered on day 1 in cycles 1-6; and
(c) prednisone or the equivalent thereof is administered on days 1-5 in cycles 1-6.
40. The method of any one of embodiments 1-39, wherein the bispecific antibody is administered subcutaneously.
41. The method of any one of embodiments 1-40, wherein rituximab is administered intravenously.
42. The method of any one of embodiments 1-41, wherein cyclophosphamide is administered intravenously.
43. The method of any one of embodiments 1-42, wherein doxorubicin is administered intravenously.
44. The method of any one of embodiments 1-43, wherein prednisone or prednisolone is administered intravenously or orally.
45. The method of any one of embodiments 1-44, wherein polatuzumab vedotin, rituximab, cyclophosphamide, doxorubicin, prednisone, and the bispecific antibody are administered sequentially.
46. The method of any one of embodiments 1-45, wherein the DLBCL is with histologically confirmed CD20+ disease.
47. The method of any one of embodiments 1-46, wherein the DLBCL is high-grade B cell lymphoma with MYC and Bcl-2 and/or Bcl-6 translocations (double-hit or triple-hit).
48. The method of any one of embodiments 1-47, wherein the DLBCL is follicular lymphoma Grade 3B.
49. The method of any one of embodiments 1-48, wherein the subject has an International Prognostic Index (IPI) score of 2-5.
50. The method of any one of embodiments 1-49, wherein the subject has not received prior therapy for DLBCL or follicular lymphoma Grade 3B.
51. The method of any one of embodiments 1-50, wherein:
   (i) the first antigen-binding region of the bispecific antibody comprises VHCDR1, VHCDR2, and VHCDR3 comprising the amino acid sequences set forth in SEQ ID NOs: 1, 2, and 3, respectively, and VLCDR1, VLCDR2, and VLCDR3 comprising the amino acid sequences set forth in SEQ ID NO: 4, the sequence GTN, and SEQ ID NO: 5, respectively; and
   (ii) the second antigen-binding region of the bispecific antibody comprises VHCDR1, VHCDR2, and VHCDR3 comprising the amino acid sequences set forth in SEQ ID NOs: 8, 9, and 10, respectively, and VLCDR1, VLCDR2, and VLCDR3 comprising the amino acid sequences set forth in SEQ ID NO: 11, the sequence DAS, and SEQ ID NO: 12, respectively.
52. The method of any one of embodiments 1-51, wherein:
   (i) the first antigen-binding region of the bispecific antibody comprises a VH region comprising the amino acid sequence of SEQ ID NO: 6, and the VL region comprising the amino acid sequence of SEQ ID NO: 7; and
   (ii) the second antigen-binding region of the bispecific antibody comprises a VH region comprising the amino acid sequence of SEQ ID NO: 13, and the VL region comprising the amino acid sequence of SEQ ID NO: 14.
53. The method of any one of embodiments 1-52, wherein the first binding arm of the bispecific antibody is derived from a humanized antibody, preferably from a full-length IgG1,λ (lambda) antibody.
54. The method of embodiment 53, wherein the first binding arm of the bispecific antibody comprises a λ light chain constant region comprising the amino acid sequence set forth in SEQ ID NO: 22.
55. The method of any one of embodiments 1-54, wherein the second binding arm of the bispecific antibody is derived from a human antibody, preferably from a full-length IgG1,κ (kappa) antibody.
56. The method of embodiment 55, wherein the second binding arm comprises a κ light chain constant region comprising the amino acid sequence set forth in SEQ ID NO: 23.
57. The method of any one of embodiments 1-56, wherein the bispecific antibody is a full-length antibody with a human IgG1 constant region.
58. The method of any one of embodiments 1-57, wherein the bispecific antibody comprises an inert Fc region.
59. The method of any one of embodiments 1-58, wherein the bispecific antibody comprises a first heavy chain and a second heavy chain, wherein in both the first and second heavy chains, the amino acids in the positions corresponding to positions L234, L235, and D265 in the human IgG1 heavy chain constant region of SEQ ID NO: 15 are F, E, and A, respectively.
60. The method of any one of embodiments 1-59, wherein the bispecific antibody comprises a first heavy chain and a second heavy chain, wherein in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
61. The method of any one of embodiments 1-60, wherein the bispecific antibody comprises a first heavy chain and a second heavy chain, wherein
   (i) in both the first and second heavy chains, the amino acids in the positions corresponding to positions L234, L235, and D265 in the human IgG1 heavy chain constant region of SEQ ID NO: 15 are F, E, and A, respectively, and
   (ii) in the first heavy chain, the amino acid in the position corresponding to F405 in the human IgG1 heavy chain constant region of SEQ ID NO: 15 is L, and wherein in the second heavy chain, the amino acid in the position corresponding to K409 in the human IgG1 heavy chain constant region of SEQ ID NO: 15 is R, or vice versa.
62. The method of embodiment 61, wherein the bispecific antibody comprises heavy chain constant regions comprising the amino acid sequences of SEQ ID NOs: 19 and 20.
63. The method of any one of embodiments 1-62, wherein the bispecific antibody comprises a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.
64. The method of any one of embodiments 1-63, wherein the bispecific antibody comprises a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.
65. The method of any one of embodiments 1-64, wherein the bispecific antibody is epcoritamab, or a biosimilar thereof.

### EXAMPLES

### Duobody®-CD3XCD20

DuoBody®-CD3xCD20 is a bsAb recognizing the T-cell antigen CD3 and the B-cell antigen CD20. DuoBody®-CD3xCD20 triggers potent T-cell-mediated killing of CD20-expressing cells. DuoBody®-CD3xCD20 has a regular IgG1 structure.

Two parental antibodies, IgG1-CD3-FEAL, a humanized IgG1λ, CD3ε-specific antibody having heavy and light chain sequences as listed in SEQ ID NOs: 24 and 25, respectively, and IgG1-CD20-FEAR, derived from human IgG1κ CD20-specific antibody 7D8 having heavy and light chain sequences as listed in SEQ ID NOs: 26 and 27, respectively, were manufactured as separate biological intermediates. Each parental antibody contains one of the complementary mutations in the CH3 domain required for the generation of DuoBody® molecules (F405L and K409R, respectively). The parental antibodies comprised three additional mutations in the Fc region (L234F, L235E and D265A; FEA). The parental antibodies were produced in mammalian Chinese hamster ovary (CHO) cell lines using standard suspension cell cultivation and purification technologies. DuoBody®-CD3xCD20 was subsequently manufactured by a controlled Fab-arm exchange (cFAE) process (Labrijn et al. 2013, Labrijn et al. 2014, Gramer et al. 2013). The parental antibodies are mixed and subjected to controlled reducing conditions. This leads to separation of the parental antibodies that, under re-oxidation, re-assemble. This way, highly pure preparations of DuoBody®-CD3xCD20 (˜93-95%) were obtained. After further polishing/purification, final product was obtained, close to 100% pure. The DuoBody®-CD3xCD20 concentration was measured by absorbance at 280 nm, using the theoretical extinction coefficient ε=1.597 mL·mg⁻¹ cm⁻¹. The final product was stored at 4° C. The product has an international proprietary name of epcoritamab.

Epcoritamab is prepared (5 mg/mL or 60 mg/mL) as a sterile clear colorless to slightly yellow solution supplied as concentrate for solution for subcutaneous (SC) injection. Epcoritamab contains buffering and tonicifying agents. All excipients and amounts thereof in the formulated product are pharmaceutically acceptable for subcutaneous injection products. Appropriate doses are reconstituted to a volume of about 1 mL for subcutaneous injection.

### Example 1: Anti-Tumor Activity of Epcoritamab in the Presence of Anti-CD20 Antibody In Vivo and in NHL Patient-Derived Samples after Anti-CD20 Treatment

The effects of the presence of an anti-CD20 antibody on the anti-tumor activity of epcoritamab in a humanized mouse xenograft model has been described in Engelberts et al., EBioMedicine 2020; 52:10265, as summarized below.

Epcoritamab was found to effectively reduce tumor growth in the xenograft model (NOD-SCID mice injected with CD20-expressing Raji-luc tumor cells and PBMCs), even in the presence of an excess of a rituximab variant with an inert Fc domain (IgG1-RTX-FEAR, containing L234F, L235E, D265A, and K409R mutations). Rituximab and IgG1-CD20, of which the CD20 arm of epcoritamab is derived, compete for CD20 binding even though they bind to a different epitope, indicating that epcoritamab is able to induce effective anti-tumor activity in the presence of circulating anti-CD20 antibodies that can compete for target binding.

Furthermore, epcoritamab induced T-cell-mediated cytotoxicity in primary DLBCL and follicular lymphoma patient biopsies taken a certain amount of time after administration of an anti-CD20 antibody (Van der Horst et al., Blood (2019) 134 (Supplement_1): 4066). Even in a biopsy taken 2 weeks after administering anti-CD20 antibody, epcoritamab was able to induce up to 40% tumor cell kill.

### Example 2: Impact of CHP on In Vitro T Cell-Mediated Cytotoxicity Induced by Epcoritamab

This experiment was performed to test the impact of CHP components separately to evaluate each component's effect on epcoritamab-induced T-cell-mediated cytotoxicity.

Briefly, T cells were pre-incubated with cyclophosphamide, doxorubicin, or prednisone for 16 hours and subsequently used in a cytotoxicity assay with epcoritamab and CD20-expressing Daudi cells as target cells (E:T ratio 2:1), in which cyclophosphamide, doxorubicin, or prednisone was added in the same concentration as during pre-incubation, respectively. Data are presented as percent viable target cells (CD4-CD8-CD22+), normalized to medium control (no Ab, no CHP component). Since doxorubicin pre-treatment affected the viability of the T cells, not all concentrations of epcoritamab could be tested.

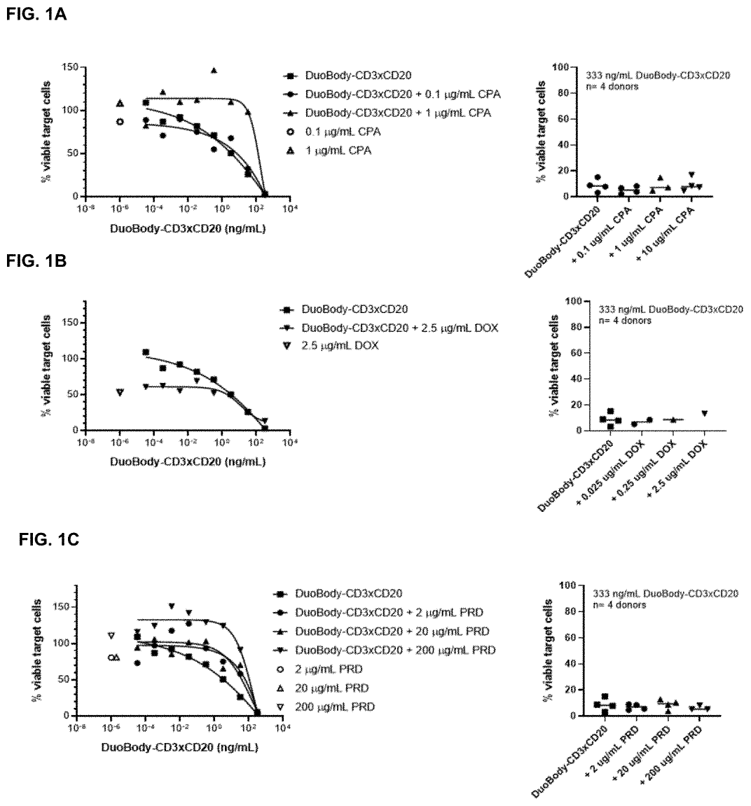

FIGS. 1A-1C show in the left panels dose response curves for DuoBody®-CD3xCD20 for a representative donor, the right panels show the response to a dose of 333 ng/ml of DuoBody®-CD3xCD20 for four different donors, with and without various concentrations of the CHP components. Pretreatment of T cells with cyclophosphamide or prednisone, respectively, did not impact T cell viability (data not shown). As said, pretreatment with doxorubicin led to a reduction in T cell viability (not shown), though the degree observed in-vitro appeared exaggerated as compared with what was observed in patients treated with R-CHOP (Oncology 2016; 91: 302-10 and Hematol Oncol 2011; 29:5-9). As shown in FIGS. 1A, 1C, T cells pretreated with cyclophosphamide (A), or prednisone (C) were able to mediate an epcoritamab-induced cytotoxic response against the CD20-expressing target cells, as shown by the dose-dependent cytotoxicity (in the left panels) and the very low percentages of viable B cells left after incubation (in the right panels). As shown in FIG. 1B, the remaining T cells pre-treated with doxorubicin were also able to mediate epcoritamab-induced cytotoxicity against target cells indicating that the remaining T cells were functional.

Taken together, the data and observations above show that epcoritamab can be combined with CHP and R-CHP, as rituximab does not interfere with epcoritamab activity, to induce highly effective anti-tumor activity against CD20-expressing target cells.

### Example 3: Phase 1b/2, Open Label Study to Evaluate Safety and Tolerability of Epcoritamab in Combination with Anti-Neoplastic Agents in Subjects with Diffuse Large B Cell Lymphoma (DLBCL)

A Phase 1b/2, open-label, multi-national, multi-center interventional trial evaluating the safety, tolerability, and preliminary efficacy of epcoritamab in combination with polatuzumab vedotin with rituximab, cyclophosphamide, doxorubicin, and prednisone (pola-R-CHP), in subjects diagnosed with DLBCL. The study will include a dose escalation phase followed by an expansion phase.

### Summary of Ongoing Clinical Trial with Epcoritamab

Epcoritamab as monotherapy is currently in a clinical trial for the treatment of R/R B-NHL (ClinicalTrials.gov Identifier: NCT03625037).

The Phase 1 study evaluating SC epcoritamab monotherapy included subjects with R/R NHL including DLBCL. The dose escalation part of the study evaluated a range of doses (12-60 mg). A full dose of 48 mg was selected as the RP2D, following one weekly priming dose of 0.16 mg and one weekly intermediate dose of 0.8 mg.

The Phase 2 study included subjects with R/R NHL, including DLBCL, and treatment-naïve DLBCL evaluated at 24 mg and 48 mg doses in an expanion phase. Clinically meaningful and compelling efficacy with epcoritamab was seen among patients with relapsed or refractory (R/R) B-NHL in the phase ½ trial (NCT03625037) trial, including deep and durable responses (overall response rate (ORR), 63%; complete response (CR] rate, 39%; median duration of response (DOR), 12 months) in a population with highly refractory large B-cell lymphoma with a manageable safety profile (n=157)(J. Clin. Oncol. Dec. 22, 2022: DOI https://doi.org/10.1200/JCO.22.01725).

An ongoing phase ½ study in high-risk patients with newly diagnosed DLBCL (NCT04663347) has shown that epcoritamab+R-CHOP has promising efficacy and a manageable safety profile in patients with International Prognostic Index (IPI) score 3-5. Among efficacy evaluable patients (n=31), ORR was 100% and complete metabolic response was 77%; cytokine release syndrome (CRS) events (n/N=17/33; 52%) were mostly low-grade and did not lead to treatment discontinuation (Falchi et al, ASCO 2022, abstract 7523).

### Objectives and Endpoints

### Primary Objectives

To characterize the safety and toxicity profiles of epcoritamab when co-administered with polatuzumab vedotin with rituximab, cyclophosphamide, doxorubicin, and prednisone (pola-R-CHP) in subjects with DLBCL.
To determine the recommended dose for further investigation of epcoritamab when co-administered with pola-R-CHP in subjects with DLBCL.

### Secondary Objectives

To evaluate the anti-NHL activity of epcoritamab when given in combination with pola-R-CHP, in subjects with DLBCL.
To characterize the pharmacokinetics of epcoritamab when given in combination with pola-R-CHP, in subjects with DLBCL.

### Exploratory Objectives

To evaluate potential mechanisms of response or resistance to therapy
To evaluate the immunogenicity of epcoritamab
To evaluate the impact on patient quality of life (QOL) through Patient-Reported Outcome Instruments (PRO), Functional Assessment of Cancer Therapy-Lymphoma (FACT-Lym) and EuroQol 5 Dimensions 5 Levels (EQ-5D-5L)

### Primary Endpoint

The primary endpoint is dose limiting toxicities (DLTs) of epcoritamab in combination with pola-R-CHP.

### Secondary Endpoints

Overall Response Rate (ORR) by Lugano 2014 criteria as assessed by investigator for epcoritamab in combination with pola-R-CHP.
Anti-lymphoma activity of epcoritamab in combination with pola-R-CHP:
Duration of response (DOR) determined per Lugano 2014 criteria as assessed by investigator
Progression free survival (PFS) determined per Lugano 2014 criteria as assessed by investigator
Complete Response (CR) rate determined per Lugano 2014 criteria as assessed by investigator
Time to response (TTR) determined per Lugano 2014 criteria as assessed by investigator
Time to next anti-lymphoma therapy (TTNT)
Rate and duration of Minimal Residual Disease (MRD) negativity
Overall survival (OS)

### Safety Endpoints

Safety and tolerability evaluations for the duration of the study include, but are not limited to:
Monitoring severity and incidence of adverse events (AE) including adverse events of special interest (AESIs)
CRS, ICANS, and CTLS
Clinical laboratory testing (hematology, chemistry, and urinalysis)
Monitoring incidence and severity of changes in laboratory values
Physical examinations
Vital signs measurements
Electrocardiogram (ECG) variables

### Pharmacokinetic Endpoints

Values for pharmacokinetic (PK) parameters, including the maximum observed plasma concentration (Cmax), the time to Cmax (Tmax), and the area under the plasma concentration versus time curve (AUC) will be determined using noncompartmental methods for epcoritamab in combination with pola-R-CHP.
Epcoritamab anti-drug antibodies (ADAs) and neutralizing ADAs in combination with pola-R-CHP.

### Study Design Overview

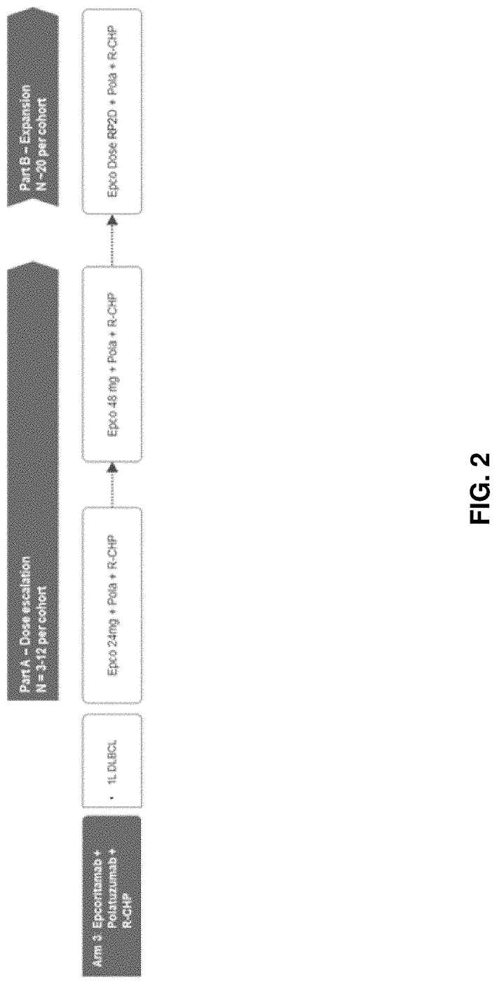

A schematic of the overall trial design is shown in FIG. 2.

The following regimens will initially be evaluated in the corresponding populations: Epcoritamab in combination with pola-R-CHP in subjects with DLBCL

### Study Treatments

Epcoritamab in combination with pola-R-CHP will be administered using a step-up dosing method: priming dose of 0.16 mg (Cycle 1 Day 1), followed by an intermediate dose of 0.8 mg (Cycle 1 Day 8), and full doses of the assigned dose level, 24 or 48 mg (Cycle 1 Day 15 onwards). Epcoritamab will be administered as a SC injection once every week (QW) in Cycles 2-4, followed by once every 3 weeks (Q3W) in Cycle 5 through Cycle 8. On the days the subject is administered epcoritamab in combination with other study drug(s), the other study drugs should be administered prior to epcoritamab. In addition, polatuzumab should be administered after prednisone and rituximab, as infusion reactions due to rituximab are more common than those for polatuzumab.
Polatuzumab 1.8 mg/kg will be administered on Day 1 of Cycles 1-6
Rituximab 375 mg/m² will be administered on Day 1 of Cycles 1-6
Cyclophosphamide 750 mg/m² will be administered on Day 1 of Cycles 1-6
Doxorubicin 50 mg/m² will be administered on Day 1 of Cycles 1-6
Prednisone 100 mg will be administered on Days 1-5 of Cycles 1-6
Epcoritamab will be administered as noted above for a total of 8 cycles

Each arm will consist of 2 phases: Dose Escalation (n up to 12 subjects for each dose level) and Expansion (n up to 20 subjects). Within each arm, subjects can only participate in one phase. Dose Escalation and Expansion phases of each arm will consist of a screening period, a treatment period, a post treatment follow-up period, safety follow-up period, and survival follow-up period.

### Dose Escalation Phase

The dose escalation phase is designed to assess the initial safety and tolerability of epcoritamab in combination with other anti-neoplastic agents.

Dose escalation will be guided by a Bayesian optimal interval (BOIN) design. The initial enrollment in a dose escalation cohort will consist of at least 3 DLT-evaluable subjects. Epcoritamab will initially be administered in combination with the corresponding anti-neoplastic agent. If acceptable safety and tolerability are observed during the DLT period, the dose of epcoritamab will be escalated to the next dose level 48 mg. The decision to de-escalate or escalate to the higher dose of epcoritamab will be made according to the BOIN design and based on the cumulative number of subjects who experience a dose limiting toxicity (DLT).

Table 2 below provides the escalation decision rule for the BOIN design with target toxicity rate of 0.25 and optimal interval of (0.204, 0.304).

TABLE 2
Dose Escalation Decision Rule

| | # Evaluable Subjects at Current Combination | | | | | | | | | |
|---|---|---|---|---|---|---|---|---|---|---|
| Action | 3 | 4 | 5 | 6 | 7 | 8 | 9 | 10 | 11 | 12 |
| Escalate if # subjects with DLT≤ | 0 | 0 | 1 | 1 | 1 | 1 | 1 | 2 | 2 | 2 |
| Stay at current dose if # subjects with | 1^{a} | 1 | — | — | 2 | 2 | 2 | 3 | 3 | 3 |
| DLT= | | | | | | | | | | |
| De-escalate if # subjects with DLT≥ | 2 | 2 | 2 | 2 | 3 | 3 | 3 | 4 | 4 | 4 |
| Eliminate if # subjects with DLT≥ | 3 | 3 | 3 | 4 | 4 | 4 | 5 | 5 | 6 | 6 |

^{a}Modified to be consistent with 3 + 3 decision rule

Dose limiting toxicities (DLTs) will be assessed during dose-escalation in order to define the recommended phase 2 dose (RP2D). For this study, the DLT evaluation period is defined as the first four weeks, i.e., 28 days after the first administration of epcoritamab.

After all subjects on a dose level have completed the DLT evaluation period, all available data will be evaluated to make a recommendation for the next dose level.

After completion of the Dose Escalation Phase, the Sponsor will review the cumulative study data and recommend a dose to be declared as the dose of epcoritamab to be used in the Dose Expansion Phase. The totality of data including safety (i.e., AEs and safety laboratory values, and observations made after the end of the DLT evaluation period), pharmacokinetics, pharmacodynamics, and preliminary efficacy will be evaluated to guide further development in the expansion phase.

### Expansion Phase

The purpose of the expansion phase is to evaluate the safety, tolerability, and preliminary clinical activity of recommended dose of epcoritamab in combination with pola-R-CHP.

In the expansion phase of the study, a total of approximately 20 subjects will be enrolled. Epcoritamab will be administered at the determined recommended Phase 2 dose (RP2D) in combination with pola-R-CHP in the same manner as was done in Dose Escalation.

A toxicity monitoring rule will be implemented after 6 subjects have been enrolled. The rule will monitor the occurrence of DLTs and will pause enrollment if the posterior probability that the DLT rate exceeds 0.25 is greater than 80%. The prior distribution for the DLT rate will be assumed to follow a beta (1.5, 4.5) distribution, reflecting a prior mean DLT rate of 0.25 and effective sample size of 6. This corresponds to the target toxicity rate (0.25) defined in the dose escalation portion and the minimum number of subjects (6) to be enrolled at the preliminary recommended dose and schedule identified for further investigation during dose escalation.

If the number of subjects experiencing a DLT exceeds the toxicity boundaries at any time after 6 subjects are enrolled, subsequent enrollment will be paused and an aggregate safety review of all available data will be performed. Based on the toxicity monitoring rule, enrollment will be paused if the number of subjects experiencing a DLT meets any of the following boundaries:
≥3 subjects of 6 subjects enrolled
≥4 subjects of 7 to 9 subjects enrolled
≥5 subjects of 10 to 12 subjects enrolled
≥6 subjects of 13 to 16 subjects enrolled
≥7 subjects of 17 to 19 subjects enrolled
≥8 subjects of 20 subjects enrolled
   Inclusion criteria
   Subjects must meet all of the following criteria in order to be included in the study:
Adult male or female, at least 18 years old.
Laboratory values meeting the following criteria within the screening period prior to the first dose of study drug:
Absolute neutrophil count (ANC)≥1.0×109/L (growth factor use is allowed if evidence of bone marrow involvement, but subject must not have received growth factor within 14 days prior to screening labs)
Hemoglobin≥8.0 g/dL (RBC transfusions permitted, but subject must not have received blood transfusions within 7 days prior to screening labs)
Platelet count≥75×109/L, or ≥50×109/L if bone marrow infiltration or splenomegaly (platelet transfusions permitted, but subject must not have received blood transfusions within 7 days prior to screening labs)
Serum aspartate transaminase (AST) or alanine transaminase (ALT) level≤3×ULN
Total bilirubin level≤1.5×ULN or ≤5×ULN for subjects with hepatic involvement of disease or of non-hepatic origin. Subjects with Gilbert's syndrome may have total bilirubin levels>1.5×ULN, but direct bilirubin must be <2×ULN
Estimated Creatinine Clearance (CrCl)≥50 mL/min (as calculated by Cockcroft-Gault Formula, modified as needed for factors such as body weight)
Prothrombin time (PT)/International normalized ratio (INR)/Activated partial thromboplastin time (aPTT)≤1.5×ULN, unless receiving anticoagulation
Subject must be able to tolerate subcutaneous injections
Subject must have available adequate fresh or paraffin-imbedded tissue at Screening

### Disease/Condition Activity

Diagnosis of DLBCL (de novo or histologically transformed from follicular lymphoma or nodal marginal zone lymphoma) with histologically confirmed CD20+ disease, inclusive of the following according to WHO 2016 classification and documented in pathology report:
DLBCL, not otherwise specified (NOS)
High-grade B cell lymphoma with MYC and BCL-2 and/or BCL-6 translocations per WHO 2016 (“double-hit” or “triple-hit”)
Note: High-grade B-cell lymphomas NOS or other double-/triple-hit lymphomas (with histologies not consistent with DLBCL) are not eligible
Follicular lymphoma Grade 3B
Subject must have no prior treatment with a bispecific antibody targeting CD3 and CD20
Subject must have 1 or more measurable disease sites:
A positron emission tomography/computed tomography (PET/CT) scan demonstrating PET-positive lesion(s)
AND
At least 1 measurable nodal lesion (long axis≥1.5 cm and short axis≥1.0 cm) or ≥1 measurable extra-nodal lesion (long axis≥1.0 cm) on CT scan or MRI
Subject must be eligible to receive and have a need for treatment initiation based on symptoms and/or disease burden as per investigator assessment.
Subject must have Eastern Cooperative Oncology Group (ECOG) performance status 0-2.
Subject has no unresolved toxicities from prior anticancer therapy, defined as having not resolved to Common Terminology Criteria for Adverse Events (CTCAE, v 5.0), Grade 1, with the exception of alopecia. Other eligibility criteria (e.g., laboratory, cardiac criteria) must also be met.
Subject has no current evidence of primary central nervous system (CNS) tumor or known CNS involvement, including leptomeningeal disease, at screening.
Subject has no history of severe allergic or anaphylactic reactions to anti-CD20 mAb therapy or known significant allergy or intolerance to any component or excipient of epcoritamab or components of study drug combination agents (e.g., lenalidomide, rituximab, etc.)
Subject must not have had autologous stem cell transplantation within 3 months prior to screening.
Subject must not have had chemotherapy, non-investigational, or investigational anti-neoplastic agents (except CD20 mAbs) within 4 weeks or 5 half-lives (whichever is shorter) prior to the first dose of epcoritamab.
Subject has no clinically significant cardiovascular disease, including:
   Myocardial infarction or stroke within 6 months prior to enrollment,
   OR
   The following conditions within 3 months prior to enrollment: unstable or uncontrolled disease/condition related to or affecting cardiac function (e.g., unstable angina, congestive heart failure, New York Heart Association Class III-IV), uncontrolled cardiac arrhythmia
   OR
   Other clinically significant electrocardiogram (ECG) abnormalities within 6 months prior to enrollment unless deemed stable and appropriately treated
   OR
   Left ventricular ejection fraction<45%.
Subject has no clinically significant liver disease, including hepatitis, current alcohol abuse, or cirrhosis.

Subject does not have active Hepatitis B Virus (HBV) or Hepatitis C Virus (HCV) infection. Subjects who are positive for hepatitis B core antibody (HBcAb), hepatitis B surface antigen (HBsAg), or hepatitis C antibody must have a negative polymerase chain reaction (PCR) result before enrollment. Those who are PCR positive will be excluded.
Subject has no known history of Human Immunodeficiency Virus (HIV) infection. Note: HIV testing does not need to be conducted at screening unless it is required per local guidelines or institutional standards.
Subject has no known active bacterial, viral, fungal, mycobacterial, parasitic, or other infection (excluding fungal infections of the nail beds) requiring intravenous (IV) therapy or IV antibiotics within 2 weeks prior to enrollment.
Subject has no evidence of significant, uncontrolled concomitant diseases that could affect compliance with the protocol or interpretation of results.
Subject has no history of other prior malignancies, except for the following:
   Malignancy treated with curative intent and with no known active disease present for ≥3 years before the first dose of study drug and felt to be at low risk for recurrence by the treating physician
   Adequately treated non-melanoma skin cancer or lentigo maligna without evidence of disease
   Adequately treated carcinoma in situ without evidence of disease
   Localized prostate cancer, post-radical prostatectomy with non-rising prostate-specific antigen (PSA) levels<0.1 ng/mL
Subject has not had radiation therapy to target lesion if only 1 target lesion is involved and no other target lesions that have not received radiation therapy can be followed, or major surgery within 4 weeks of enrollment.
Subject has no Grade>1 neuropathy.
Subject must not have active tuberculosis (TB) or history of completed treatment for active TB within the past 12 months.
   Note: Interferon gamma release assay (IGRA) testing does not need to be performed at screening unless active or latent tuberculosis is suspected. For subjects with positive IGRA, active pulmonary tuberculosis must be excluded with clinical evaluation and radiologic imaging. Subjects with positive IGRA and no evidence of active disease may be enrolled after treatment for latent tuberculosis infection (recommendation isoniazid monotherapy for total of 6 months) has been initiated.
Subject has no evidence of cytomegalovirus (CMV) viremia (defined as any positive level above the lower limit of detection) at screening.
Subject has no current autoimmune disease requiring immunosuppressive therapy except for up to 20 mg prednisone daily (or equivalent).
Subject has no life-threatening illness, medical condition, or organ system dysfunction that, in the Investigator's opinion, could compromise the subject's safety or put the study outcomes at undue risk.
Subject has no current seizure disorder requiring therapy.
Subject has no known active SARS-CoV-2 infection. If a subject has signs/symptoms suggestive of SARS-CoV-2 infection or have recent known exposure to someone with SARS-CoV infection, they should undergo molecular (e.g., PCR) testing or 2 negative antigen test results at least 24 hours apart to rule out SARS-CoV-2 infection.
Subjects who do not meet SARS-CoV-2 infection eligibility criteria must be screen failed and may only rescreen after they meet the following SARS-CoV-2 infection viral clearance criteria:
   At least 10 days since first positive test result have passed in asymptomatic patients or at least 10 days since recovery, defined as resolution of fever without use of antipyretics and improvement in symptoms.
Subject must not have had major surgery within 4 weeks of the first dose of study drug.

### Additional Eligibility Criteria:

Subject must have newly diagnosed, treatment-naïve (not including prior treatments for indolent lymphoma that has transformed) DLBCL.
Subject must be suitable for treatment with polatuzumab, rituximab, cyclophosphamide, doxorubicin, and prednisone in the opinion of the investigator.

### Dose-Limiting Toxicities

### Dose-Limiting Toxicities

A DLT-evaluable subject in the dose escalation phase is defined as a subject who has received at least 3 doses of epcoritamab at the assigned dose level in the first cycle or experiences a DLT during the 28-day period after the first dose of epcoritamab.

The DLT evaluation period is defined as the first 4 weeks, i.e., 28 days after the first administration of epcoritamab, provided the subject has received at least 3 epcoritamab doses during this period.

The following will qualify for a DLT, unless the Investigator can attribute the event to a clearly identifiable cause such as underlying illness, disease progression/relapse, other concurrent illness, or from concomitant therapy.
Grade 5 toxicity
   CRS grading according to American Society for Transplantation and Cellular Therapy (ASTCT) criteria and DLT criteria for CRS
      Grade 4 CRS or ICANS according to ASTCT criteria
      Grade 3 CRS or ICANS according to ASTCT criteria which has NOT improved to
      Grade≤2 or resolved (Grade 0) within 48 hours
   Neutropenia Grade 4 lasting>7 days Graded by CTCAE.
   Febrile neutropenia Grade≥3 lasting>2 days Graded by CTCAE.
   Thrombocytopenia Grade 4 lasting>7 days Graded by CTCAE.
   Non-hematological toxicity Grade 3 or higher as Graded by CTCAE, excluding the following:
      Grade 3 fever (>40.0° C. for <24 hours)
      Grade 3 hypotension (resolving within 24 hours)
      Laboratory values out of normal range which do not have any clinical consequence, are clinically transient, isolated in nature and which resolve within 7 days (this includes electrolyte abnormalities that respond to medical intervention)
      AST and/or ALT Grade 3 returned to Grade 1 or baseline within 7 days.
      Grade 3 nausea that responds to optimal antiemetic treatment within 3 days.
      Grade 3 vomiting that responds to optimal antiemetic treatment within 3 days.
      Grade 3 diarrhea that responds to optimal antidiarrheal treatment within 3 days.
      Grade 3 fatigue/asthenia when fatigue/asthenia was present at baseline or that lasts for <14 days after the last administration of epcoritamab.
      Other Grade 3 toxicity related to prior chemotherapy that was present at baseline (Grade 1 or 2) and returned to baseline within 7 days.
      Alopecia (no grading)

Frequent laboratory monitoring of complete blood count including differential should be initiated to document start and resolution of hematological AEs. All AEs occurring during the defined DLT evaluation period will be assessed according to the criteria above. All AEs, including those not qualifying for a DLT, will be monitored and included in the evaluation of the toxicity profile of epcoritamab unless the event is clearly determined to be unrelated to epcoritamab.

### Adverse Events of Special Interest

The following adverse events of special interest will be monitored during the study:
Cytokine Release Syndrome (CRS)
Clinical Tumor Lysis Syndrome (CTLS)
Immune Cell-Associated Neurotoxicity Syndrome (ICANS)

### CRS Prophylaxis and Premedication

Premedication with corticosteroids, antihistamines, and antipyretics is mandatory as described in the Operations Manual, Section 3.4. For the first four doses of epcoritamab, premedication with antihistamines, antipyretics, and corticosteroids are mandatory; and an additional 3 days of corticosteroids are required following each of these first 4 doses to prevent/reduce the severity of symptoms from potential CRS. For the first 4 doses of epcoritamab, the subject must perform self-administered oral temperature monitoring 3 times a day (approximately every 6-8 hours during waking hours) for the first 4 days post epcoritamab administration. These temperature checks are to ensure that fever, an early sign of CRS, has not developed. For administration of epcoritamab beyond the fourth dose (i.e., the second full dose), CRS prophylaxis with corticosteroids is optional, unless CRS Grade 2 or higher occurs, in which case CRS prophylaxis should continue until an epcoritamab dose is given without subsequent CRS. Premedication corticosteroid administration can be either IV or PO with the recommended dose or equivalent.

### Study Assessments

### Disease Response and Progressive Disease Assessment

On-treatment assessment: Response at on-treatment time-points should be read according to Lugano Classification for patients showing CR, PR, and SD. For patients showing PD according to Lugano Classification, further evaluation should be performed to see if the subject can be considered to have IR (according to LYRIC).

### Lugano Response Criteria for Malignant Lymphoma

### Target and Non-Target Lesions

Target lesions should consist of up to six of the largest dominant nodes, nodal masses, or other lymphomatous lesions that are measurable in two diameters and should preferably be from different body regions representative of the subject's overall disease burden, including mediastinal and retroperitoneal disease, where applicable. At baseline, a measurable node must be greater than 15 mm in longest diameter (longest transverse diameter of a lesion; LDi). Measurable extranodal disease may be included in the six representative target lesions. At baseline, measurable extranodal lesions should be greater than 10 mm in LDi.

All other lesions (including nodal, extra-nodal, and assessable disease) should be followed as non-target lesions (e.g., cutaneous, GI, bone, spleen, liver, kidneys, pleural or pericardial effusions, ascites, bone, bone marrow).

### Split Lesions and Confluent Lesions

Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate PD. The LDi and smallest diameter (shortest axis perpendicular to the LDi; SDi) are no longer needed to determine progression.

TABLE 3
Lugano Response Criteria for Malignant Lymphoma

| Response | Site | PET-CT-Based Response | CT-Based Response |
|---|---|---|---|
| Complete | | Complete Metabolic Response | Complete Radiologic |
| Response | | | Response (all of the |
| | | | following) |
| | Lymph nodes | Score 1, 2, or 3 with or without a | Target nodes/nodal masses |
| | and extralymphatic sites | residual mass on 5PS. | must regress to ≤1.5 cm |
| | | It is recognized that in Waldeyer's | in LDi. No extralymphatic |
| | | ring or extranodal sites with high | sites of disease |
| | | physiologic uptake or with | |
| | | activation within spleen or | |
| | | marrow (e.g., with chemotherapy | |
| | | or myeloid colony-stimulating | |
| | | factors), uptake may be greater | |
| | | than normal mediastinum and/or | |
| | | liver. In this circumstance, | |
| | | complete metabolic response may | |
| | | be inferred if uptake at sites of | |
| | | initial involvement is no greater | |
| | | than surrounding normal tissue | |
| | | even if the tissue has high | |
| | | physiologic uptake. | |
| | Non-measured | Not applicable | Absent |
| | lesion | | |
| | Organ | Not applicable | Regress to normal |
| | enlargement | | |
| | New lesions | None | None |
| | Bone marrow | No evidence of FDG-avid disease | Normal by morphology; if |
| | | in marrow | indeterminate, IHC negative |
| Partial | | Partial Metabolic Response | Partial Remission (all of the |
| Response | | | following) |
| | Lymph nodes and | Score 4 or 5² with reduced uptake | ≥50% decrease in SPD of up |
| | extralymphatic | compared with baseline and | to 6 target measurable nodes |
| | sites | residual mass(es) of any size | and extranodal sites |
| | | At interim, these findings suggest | When a lesion is too small to |
| | | responding disease. At end of | measure on CT, assign |
| | | treatment, these findings indicate | 5 mm × 5 mm as the default |
| | | residual disease. | value When no longer visible, |
| | | | 0 × 0 mm. For a node >5 |
| | | | mm × 5 mm, but smaller than |
| | | | normal, use actual |
| | | | measurement for calculation |
| | Non-measured | Not applicable | Absent/normal, regressed, but |
| | lesion | | no increase |
| | Organ | Not applicable | Spleen must have regressed |
| | enlargement | | by >50% in length beyond |
| | | | normal |
| | New lesions | None | None |
| | Bone marrow | Residual uptake higher than | Not applicable |
| | | uptake in normal marrow but | |
| | | reduced compared with baseline | |
| | | (diffuse uptake compatible with | |
| | | reactive changes from | |
| | | chemotherapy allowed). If there | |
| | | are persistent focal changes in the | |
| | | marrow in the context of a nodal | |
| | | response, consideration should be | |
| | | given to further evaluation with | |
| | | MRI or biopsy or an interval scan. | |
| No | | No metabolic response | Stable disease |
| response or | Target | Score 4 or 5² with no significant | <50% decrease from baseline |
| Stable | nodes/nodal | change in FDG uptake from | in SPD of up to 6 dominant, |
| disease | masses, | baseline at interim or end of | measurable nodes and |
| | extranodal | treatment | extranodal sites; no criteria for |
| | lesions | | progressive disease are met |
| | Non-measured lesion | Not applicable | No increase consistent with |
| | | | progression |
| | Organ | Not applicable | No increase consistent with |
| | enlargement | | progression |
| | New lesions | None | None |
| | Bone marrow | No change from baseline | Not applicable |
| Progressive | | Progressive metabolic disease | Progressive disease requires |
| disease | | | at least 1 of the following |
| | Individual | Score 4 or 5² with an increase in | PPD progression: |
| | target | intensity of uptake from baseline | An individual node/lesion |
| | nodes/nodal | and/or | must be abnormal with: |
| | masses, | New FDG-avid foci consistent | LDi > 1.5 cm and |
| | extranodal | with lymphoma at interim or end- | Increase by ≥50% from |
| | lesions | of-treatment assessment | PPD nadir and |
| | | | An increase in LDi or SDi |
| | | | from nadir |
| | | | 0.5 cm for lesions ≤ 2 cm |
| | | | 1.0 cm for lesions > 2 cm |
| | | | In the setting of splenomegaly |
| | | | (>13 cm), the splenic length |
| | | | must increase by >50% of the |
| | | | extent of its prior increase |
| | | | beyond baseline (e.g., a 15-cm |
| | | | spleen must increase to ≥16 |
| | | | cm). If no prior |
| | | | splenomegaly, must increase |
| | | | by at least 2 cm from baseline. |
| | | | New or recurrent |
| | | | splenomegaly |
| | Non-measured lesions | None | New or clear progression of |
| | | | pre-existing non-measured |
| | | | lesions |
| | New lesions | New FDG-avid foci consistent | Regrowth of previously |
| | | with lymphoma rather than | resolved lesions |
| | | another etiology (e.g., infection, | A new node >1.5 cm in any |
| | | inflammation); if uncertain | axis |
| | | regarding etiology of new lesions, | A new extranodal site >1.0 cm |
| | | biopsy or interval scan may be | in any axis; if <1.0 cm in any |
| | | considered | axis, its presence must be |
| | | | unequivocal and must be |
| | | | attributable to lymphoma |
| | | | Assessable disease of any size |
| | | | unequivocally attributable to |
| | | | lymphoma |
| | Bone marrow | New or recurrent FDG-avid foci | New or recurrent involvement |

5PS = 5-point scale; CT = computed tomography; FDG = fluorodeoxyglucose; IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.
1. A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment).
Measured dominant (target) lesions: Up to six of the largest dominant nodes, nodal masses, and extranodal lesions selected to be clearly measurable in two diameters.
Nodes should preferably be from disparate regions of the body and should include, where applicable, mediastinal, and retroperitoneal areas.
Non-nodal lesions include those in solid organs (e.g., liver, spleen, kidneys, lungs), gastrointestinal involvement, cutaneous lesions, or those noted on palpation.
Non-measured lesions: Any disease not selected as measured, dominant disease and truly assessable disease should be considered not measured.
These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging.
In Waldeyer's ring or in extranodal sites (e.g., GI tract, liver, bone marrow), FDG uptake may be greater than in the mediastinum with complete metabolic response but should be no higher than surrounding normal physiologic uptake (e.g., with marrow activation as a result of chemotherapy or myeloid growth factors).
2. PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions; × = new areas of uptake unlikely to be related to lymphoma.
Source: Cheson B D, Fisher R, Barrington S F, et al. Recommendations for initial evaluation, staging, and response assessment of Hodgkin and non-Hodgkin lymphoma: the Lugano classification. J Clin Oncol. 2014; 32: 3059-68.

### Lymphoma Response to Immunomodulatory Therapy Criteria (LYRIC)

Clinical studies have shown that cancer immunotherapies may result in early apparent radiographic progression (including the appearance of new lesions), followed by a delayed response. As this initial increase in tumor size might be caused by immune-cell infiltration in the setting of a T-cell response, this progression may not be indicative of true disease progression and is therefore called “pseudoprogression”.

The association of epcoritamab (GEN3013; DuoBody®-CD3xCD20) with pseudoprogression is currently unknown, but its mechanism of action implies that pseudoprogression is to be expected.

The current Lugano response assessment criteria does not take pseudoprogression into account, and there is a significant risk of premature discontinuation of a potentially efficacious immunomodulatory drug following the observation of an atypical response. Atypical responses are characterized either by the early progression of existing lesions, later followed by response, or by the development of new lesions, with or without tumor shrinkage elsewhere.

LYRIC is a modification of the Lugano response assessment criteria, which has been adapted to immune-based therapies, and it implements a new, mitigating response category: the “indeterminate response” (IR) designation. This IR designation was introduced to potentially identify “atypical response” cases until confirmed as flare/pseudoprogression or true PD by either biopsy or subsequent imaging. LYRIC and the Lugano criteria will be assessed in this study.

### Indeterminate Response (IR) Category

A subject who shows PD according Lugano Classification⁷ will be considered to have IR in 1 or more of the 3 following circumstances.
IR (1): Increase in overall tumor burden (as assessed by sum of the product of the diameters [SPD]) of ≥50% of up to 6 target lesions in the first 12 weeks of therapy, without clinical deterioration.
IR (2): Appearance of new lesions or growth of one or more existing lesion(s)≥50% at any time during treatment; occurring in the context of lack of overall progression (SPD<50% increase) of overall tumor burden, as measured by SPD of up to 6 lesions at any time during the treatment.
IR (3): Increase in FDG uptake of 1 or more lesion(s) without a concomitant increase in lesion size or number.
Screening assessment: At screening, the FDG-PET/CT and diagnostic CT or MRI scans should be read according to Lugano Classification, as detailed above.
On-treatment assessment: Response at on-treatment time-points should be read according to Lugano Classification for patients showing CR, PR, and SD. For patients showing PD according to Lugano Classification, further evaluation should be performed to see if the subject can be considered to have IR (according to LYRIC).

### Statistical Analyses for Efficacy

Descriptive statistics and subject listings will be used to summarize the data for each epcoritamab dose level (24 mg and 48 mg). For continuous variables, number of observations, means, standard deviations, medians, and ranges will be used. For categorical variables, frequency and percentage will be summarized. For time-to-event endpoints, Kaplan-Meier estimates will be provided.

### Summary and Analysis of Key Secondary Efficacy Endpoints

Overall response rate (ORR) is defined as the proportion of subjects who achieved best overall response of CR or PR determined by Lugano 2014 criteria as assessed by investigators. Point estimate along with 95% exact confidence interval (CI) will be provided for each arm.

Duration of response (DOR) is defined for subjects who achieved best overall response of CR or PR (‘responders’), as the time in months from initial CR/PR to the earliest occurrence of disease progression determined by Lugano 2014 criteria as assessed by investigator, or death from any cause. Surviving responders without radiographic disease progression will be censored at the time of the last adequate disease assessment.

Number of responders, number of DOR events and the earliest contributing event (disease progression or death) will be summarized by arm. The Kaplan-Meier method will be used to estimate the distribution of DoR for each arm.

Progression-free survival (PFS) is defined for subjects in all arms, as the time in months from the first dose of study drug to the earliest occurrence of disease progression determined by Lugano 2014 criteria as assessed by investigator, or death from any cause. Surviving subjects without disease progression will be censored at the time of the last adequate disease assessment. Surviving subjects without post-baseline disease assessment will be censored at the date of first dose of study drug.

Number of PFS events and the earliest contributing event (disease progression or death) will be presented by arm. The Kaplan-Meier method will be used to estimate the distribution of PFS.

Complete response rate is defined as the proportion of subjects who achieved best overall response of CR determined by Lugano 2014 criteria as assessed by investigator. Point estimate along with 95% exact confidence interval (CI) will be provided for each arm.
Time to response (TTR) is defined for subjects who achieved best overall response of CR or PR (‘responders’) determined by Lugano 2014 criteria as assessed by investigator, as the time in months from first dose of study drug to initial CR/PR.
Number of responders along with descriptive summaries of TTR will be provided for each arm.
Overall survival (OS) is defined for subjects in all arms, as the time in months from first dose of epcoritamab to death from any cause. Subjects that are still alive at the end of the study or at the time of the analysis will be censored at last known alive date.
Number of deaths, and Kaplan-Meier estimates of distribution of OS will be provided.

### Statistical Analyses for Safety

Safety and tolerability of epcoritamab in combination with other agents will be assessed by evaluation of study drug exposure, incidence of dose interruptions, reductions, delays and discontinuations, AEs including AESIs, SAEs, deaths and changes in adverse events and vital signs parameters.

Treatment-emergent AEs will be summarized by Preferred Terms within a System Organ Class according to the Medical Dictionary for Regulatory Activities. The number and percentage of subjects experiencing a DLT will be summarized. Additional details will be provided in the SAP.

Where applicable, blood chemistry and hematology laboratory determinations will be categorized according to the NCI CTCAE and summarized. Additional details will be provided in the SAP.

### Statistical Analyses for Pharmacokinetics

Plasma concentrations for epcoritamab along with PK parameter values will be tabulated for drug within each cohort. Summary statistics will be computed by sampling time for PK concentrations and by cycle and/or visits for PK parameters. Results for epcoritamab ADA (and nAb, if applicable) will be summarized. Additional exploratory analyses may be conducted as deemed appropriate.

### Preliminary Results

Dose Escalation (Epcoritamab 24 mg+Pola-R-CHP)
# of Subjects Enrolled: 8
# of Subjects projected with at least 1 post-baseline efficacy assessment: 8
# of Subjects with available post-baseline efficacy assessment: 4
   ORR=100% (4/4)
   CRR=75% (¾)
Dose Escalation (Epcoritamab 48 mg+Pola-R-CHP)
# of Subjects Enrolled: 4
# of Subjects projected with at least 1 post-baseline efficacy assessment: 0
# of Subjects with available post-baseline efficacy assessment: 0

TABLE 4
Summary of Sequences

| SEQ | | |
|---|---|---|
| ID | Description | Sequence |
| 1 | huCD3 VH CDR1 | GFTFNTYA |
| 2 | huCD3 VH CDR2 | IRSKYNNYAT |
| 3 | huCD3 VH CDR3 | VRHGNFGNSYVSWFAY |
| 4 | huCD3 VL CDR1 | TGAVTTSNY |
| — | huCD3 VL CDR2 | GTN |
| 5 | huCD3 VL CDR3 | ALWYSNLWV |
| 6 | huCD3 VH1 | EVKLVESGGGLVQPGGSLRLSCAASGFTFNTYAMNWVRQAPGKGLE |
| | | WVARIRSKYNNYATYYADSVKDRFTISRDDSKSSLYLQMNNLKTEDTA |
| | | MYYCVRHGNFGNSYVSWFAYWGQGTLVTVSS |
| 7 | huCD3 VL1 | QAVVTQEPSFSVSPGGTVTLTCRSSTGAVTTSNYANWVQQTPGQAF |
| | | RGLIGGTNKRAPGVPARFSGSLIGDKAALTITGAQADDESIYFCALWYS |
| | | NLWVFGGGTKLTVL |
| 8 | VH CD20 - 7D8 CDR1 | GFTFHDYA |
| 9 | VH CD20 - 7D8 CDR2 | ISWNSGTI |
| 10 | VH CD20 - 7D8 CDR3 | AKDIQYGNYYYGMDV |
| 11 | VL CD20 - 7D8 CDR1 | QSVSSY |
| - | VL CD20 - 7D8 CDR2 | DAS |
| 12 | VL CD20 - 7D8 CDR3 | QQRSNWPIT |
| 13 | VH CD20 - 7D8 | EVQLVESGGGLVQPDRSLRLSCAASGFTFHDYAMHWVRQAPGKGLE |
| | | WVSTISWNSGTIGYADSVKGRFTISRDNAKNSLYLQMNSLRAEDTAL |
| | | YYCAKDIQYGNYYYGMDVWGQGTTVTVSS |
| 14 | VL CD20 - 7D8 | EIVLTQSPATLSLSPGERATLSCRASQSVSSYLAWYQQKPGQAPRLLIY |
| | | DASNRATGIPARFSGSGSGTDFTLTISSLEPEDFAVYYCQQRSNWPITF |
| | | GQGTRLEIK |
| 15 | IgG1 heavy chain | ASTKGPSVFPLAPSSKSTSGGTAALGCLVKDYFPEPVTVSWNSGALTS |
| | constant region - WT | GVHTFPAVLQSSGLYSLSSVVTVPSSSLGTQTYICNVNHKPSNTKVDK |
| | (amino acids positions | RVEPKSCDKTHTCPPCPAPELLGGPSVFLFPPKPKDTLMISRTPEVTCV |
| | 118-447 according to | VVDVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | CH3 region italics | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 16 | IgG1-LFLEDA heavy | ASTKGPSVFPLAPSSKSTSGGTAALGCLVKDYFPEPVTVSWNSGALTS |
| | chain constant region | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering). | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 17 | IgG1 F405L | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 18 | IgG1-K409R | ASTKGPSVFPLAPSSKSTSGGTAALGCLVKDYFPEPVTVSWNSGALTS |
| | (amino acids positions | GVHTFPAVLQSSGLYSLSSVVTVPSSSLGTQTYICNVNHKPSNTKVDK |
| | 118-447 according to | RVEPKSCDKTHTCPPCPAPELLGGPSVFLFPPKPKDTLMISRTPEVTCV |
| | EU numbering) | VVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 19 | IgG1 -LFLEDA-F405L | ASTKGPSVFPLAPSSKSTSGGTAALGCLVKDYFPEPVTVSWNSGALTS |
| | (FEAL) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FLLYSKLTVDKSRWQQGNVFSCSVMHEALHNHYTQKSLSLSPG |
| 20 | IgG1 -LFLEDA-K409R | ASTKGPSVFPLAPSSKSTSGGTAALGCLVKDYFPEPVTVSWNSGALTS |
| | (FEAR) | GVHTFPAVLQSSGLYSLSSVVTVPSSSLGTQTYICNVNHKPSNTKVDK |
| | (amino acids positions | RVEPKSCDKTHTCPPCPAPEFEGGPSVFLFPPKPKDTLMISRTPEVTCV |
| | 118-447 according to | VVAVSHEDPEVKFNWYVDGVEVHNAKTKPREEQYNSTYRVVSVLTV |
| | EU numbering) | LHQDWLNGKEYKCKVSNKALPAPIEKTISKAKGQPREPQVYTLPPSRE |
| | | EMTKNQVSLTCLVKGFYPSDIAVEWESNGQPENNYKTTPPVLDSDGS |
| | | FFLYSRLTVDKSRWQQGNVFSCSVMHEALHNHYTQKSLSLSPG |
| 21 | IgG1 CH3 region | GQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALH |
| | | NHYTQKSLSLSPG |
| 22 | Constant region | GQPKAAPSVTLFPPSSEELQANKATLVCLISDFYPGAVTVAWKADSSP |
| | human lambda LC | VKAGVETTTPSKQSNNKYAASSYLSLTPEQWKSHRSYSCQVTHEGST |
| | | VEKTVAPTECS |
| 23 | Constant region | RTVAAPSVFIFPPSDEQLKSGTASVVCLLNNFYPREAKVQWKVDNAL |
| | human kappa LC | QSGNSQESVTEQDSKDSTYSLSSTLTLSKADYEKHKVYACEVTHQGLS |
| | | SPVTKSFNRGEC |
| 24 | huCD3-LFLEDA-F405L | EVKLVESGGGLVQPGGSLRLSCAASGFTFNTYAMNWVRQAPGKGLE |
| | (FEAL) | WVARIRSKYNNYATYYADSVKDRFTISRDDSKSSLYLQMNNLKTEDTA |
| | heavy chain | MYYCVRHGNFGNSYVSWFAYWGQGTLVTVSSASTKGPSVFPLAPSS |
| | | KSTSGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTCPPC |
| | | PAPEFEGGPSVFLFPPKPKDTLMISRTPEVTCVVVAVSHEDPEVKFNW |
| | | YVDGVEVHNAKTKPREEQYNSTYRVVSVLTVLHQDWLNGKEYKCKV |
| | | SNKALPAPIEKTISKAKGQPREPQVYTLPPSREEMTKNQVSLTCLVKGF |
| | | YPSDIAVEWESNGQPENNYKTTPPVLDSDGSFLLYSKLTVDKSRWQQ |
| | | GNVFSCSVMHEALHNHYTQKSLSLSPG |
| 25 | huCD3 VL + CL light | QAVVTQEPSFSVSPGGTVTLTCRSSTGAVTTSNYANWVQQTPGQAF |
| | chain | RGLIGGTNKRAPGVPARFSGSLIGDKAALTITGAQADDESIYFCALWYS |
| | | NLWVFGGGTKLTVLGQPKAAPSVTLFPPSSEELQANKATLVCLISDFY |
| | | PGAVTVAWKADSSPVKAGVETTTPSKQSNNKYAASSYLSLTPEQWKS |
| | | HRSYSCQVTHEGSTVEKTVAPTECS |
| 26 | CD20-7D8-LFLEDA- | EVQLVESGGGLVQPDRSLRLSCAASGFTFHDYAMHWVRQAPGKGLE |
| | K409R (FEAR) | WVSTISWNSGTIGYADSVKGRFTISRDNAKNSLYLQMNSLRAEDTAL |
| | heavy chain | YYCAKDIQYGNYYYGMDVWGQGTTVTVSSASTKGPSVFPLAPSSKST |
| | | SGGTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPCPA |
| | | PEFEGGPSVFLFPPKPKDTLMISRTPEVTCVVVAVSHEDPEVKFNWYV |
| | | DGVEVHNAKTKPREEQYNSTYRVVSVLTVLHQDWLNGKEYKCKVSN |
| | | KALPAPIEKTISKAKGQPREPQVYTLPPSREEMTKNQVSLTCLVKGFYP |
| | | SDIAVEWESNGQPENNYKTTPPVLDSDGSFFLYSRLTVDKSRWQQG |
| | | NVFSCSVMHEALHNHYTQKSLSLSPG |
| 27 | CD20 - 7D8 VL + CL | EIVLTQSPATLSLSPGERATLSCRASQSVSSYLAWYQQKPGQAPRLLIY |
| | light chain | DASNRATGIPARFSGSGSGTDFTLTISSLEPEDFAVYYCQQRSNWPITF |
| | | GQGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| 28 | Human CD3 (epsilon) | MQSGTHWRVLGLCLLSVGVWGQDGNEEMGGITQTPYKVSISGTTVI |
| | | LTCPQYPGSEILWQHNDKNIGGDEDDKNIGSDEDHLSLKEFSELEQSG |
| | | YYVCYPRGSKPEDANFYLYLRARVCENCMEMDVMSVATIVIVDICITG |
| | | GLLLLVYYWSKNRKAKAKPVTRGAGAGGRQRGQNKERPPPVPNPDY |
| | | EPIRKGQRDLYSGLNQRRI |
| 29 | Human CD20 | MTTPRNSVNGTFPAEPMKGPIAMQSGPKPLFRRMSSLVGPTQSFFM |
| | | RESKTLGAVQIMNGLFHIALGGLLMIPAGIYAPICVTVWYPLWGGIM |
| | | YIISGSLLAATEKNSRKCLVKGKMIMNSLSLFAAISGMILSIMDILNIKIS |
| | | HFLKMESLNFIRAHTPYINIYNCEPANPSEKNSPSTQYCYSIQSLFLGILS |
| | | VMLIFAFFQELVIAGIVENEWKRTCSRPKSNIVLLSAEEKKEQTIEIKEEV |
| | | VGLTETSSQPKNEEDIEIIPIQEEEEEETETNFPEPPQDQESSPIENDSSP |

Bold and underlined are F; E; A; L and R, corresponding to positions 234 and 235; 265; 405 and 409, respectively, said positions being in accordance with EU-numbering. In variable regions, said CDR regions that were annotated in accordance with IMGT definitions are underlined.

1. A method of treating diffuse large B-cell lymphoma (DLBCL) in a human subject, the method comprising administering to the subject of a combination comprising epcoritamab or a biosimilar thereof, polatuzumab vedotin, rituximab, cyclophosphamide, doxorubicin and prednisone or prednisolone in 21-day cycles, wherein
(a) epcoritamab or biosimilar thereof is administered subcutaneously, wherein
(i) a priming dose is administered on day 1 of the first 21-day cycle, an intermediate dose is administered on day 8 of the first 21-day cycle, and a full dose of 24 or 48 mg on day 15 of the first 21-day cycle, wherein the priming dose and intermediate dose are at a lower dose as compared with the full dose,
(ii) a full dose of 24 or 48 mg on days 1, 8 and 15 of the second to fourth 21-day cycle, and
(iii) a full dose of 24 or 48 mg on day 1 of each subsequent 21-day cycle;
(b) polatuzumab vedotin is administered intravenously at a dose of 1.8 mg/kg on day 1 of each 21-day cycle;
(c) rituximab is administered intravenously at a dose of 375 mg/m² on day 1 of each 21-day cycle;
(d) cyclophosphamide is administered intravenously at a dose of 750 mg/m² day 1 of each 21-day cycle;
(e) doxorubicin is administered intravenously at a dose of 50 mg/m² on day 1 of each 21-day cycle; and
(f) prednisone or prednisolone is administered intravenously or orally at a dose of 100 mg/day on days 1-5 of each 21-day cycle;
wherein administration of the combination in 21-day cycles continues until the subject exhibits a complete response (CR) or until progressive disease develops or unacceptable toxicity occurs.

2. The method of claim 1, wherein the epcoritamab or biosimilar thereof is administered at a full dose of 24 mg.

3. The method of claim 1, wherein the epcoritamab or biosimilar thereof is administered at a full dose of 48 mg.

4-6. (canceled)

7. The method of claim 1, wherein the administration of the full dose of epcoritamab or biosimilar thereof is performed on day 1 for at least cycles 4-8.

8-10. (canceled)

11. The method of claim 1, wherein the priming dose of epcoritamab is 0.16 mg.

12-13. (canceled)

14. The method of claim 1, wherein the intermediate dose is 0.8 mg.

15-16. (canceled)

17. The method of claim 1, wherein the administration of polatuzumab vedotin is performed for six 21-day cycles.

18-24. (canceled)

25. The method of claim 1, wherein the administration of cyclophosphamide is performed for six 21-day cycles.

26-28. (canceled)

29. The method of claim 1, wherein the administration of doxorubicin is performed for six 21-day cycles.

30-33. (canceled)

34. The method of claim 1, wherein prednisone or prednisolone is administered for six 21-day cycles.

35-45. (canceled)

46. The method of claim 1, wherein the DLBCL is with histologically confirmed CD20+ disease.

47. The method of claim 46, wherein the DLBCL is high-grade B cell lymphoma with MYC and Bcl-2 and/or Bcl-6 translocations (double-hit or triple-hit).

48. The method of claim 47, wherein the DLBCL is follicular lymphoma Grade 3B.

49. The method of claim 1, wherein the subject has an International Prognostic Index (IPI) score of 2-5.

50. The method of claim 1, wherein the subject has not received prior therapy for DLBCL or follicular lymphoma Grade 3B.

51-62. (canceled)

63. The method of claim 1, wherein the method comprises administering a biosimilar of epcoritamab comprising a heavy chain and a light chain comprising the amino acid sequences set forth in SEQ ID NOs: 24 and 25, respectively, and a heavy chain and a light chain comprising the amino acid sequences set forth in SEQ ID NOs: 26 and 27, respectively.

64. The method of claim 1, wherein the method comprises administering a biosimilar of epcoritamab comprising a heavy chain and a light chain consisting of the amino acid sequence of SEQ ID NOs: 24 and 25, respectively, and a heavy chain and a light chain consisting of the amino acid sequence of SEQ ID NOs: 26 and 27, respectively.

65. (canceled)

66. The method of claim 1, wherein the method comprises administration of epcoritamab.

67. A method of treating diffuse large B-cell lymphoma (DLBCL) in a human subject, the method comprising administering to the subject of a combination comprising epcoritamab, polatuzumab vedotin, rituximab, cyclophosphamide, doxorubicin and prednisone or prednisolone in 21-day cycles, wherein
(a) epcoritamab is administered subcutaneously
(i) at a priming dose of 0.16 mg on day 1 of the first 21-day cycle, at an intermediate dose of 0.8 mg on day 8 of the first 21-day cycle, and at a full dose of 24 on day 15 of the first 21-day cycle,
(ii) at a full dose of 24 mg on days 1, 8 and 15 of the second to fourth 21-day cycle, and
(iii) at a full dose of 24 mg on day 1 of each subsequent 21-day cycle;
(b) polatuzumab vedotin is administered at a dose of 1.8 mg/kg on day 1 of each 21-day cycle;
(c) rituximab is administered intravenously at a dose of 375 mg/m² on day 1 of each 21-day cycle;
(d) cyclophosphamide is administered intravenously at a dose of 750 mg/m² day 1 of each 21-day cycle;
(e) doxorubicin is administered intravenously at a dose of 50 mg/m² on day 1 of each 21-day cycle; and
(f) prednisone or prednisolone is administered intravenously or orally at a dose of 100 mg/day on days 1-5 of each 21-day cycle;
wherein administration of the combination in 21-day cycles continues until the subject exhibits a complete response (CR) or until progressive disease develops or unacceptable toxicity occurs.

68. A method of treating diffuse large B-cell lymphoma (DLBCL) in a human subject, the method comprising administering to the subject of a combination comprising epcoritamab, polatuzumab vedotin, rituximab, cyclophosphamide, doxorubicin and prednisone or prednisolone in 21-day cycles, wherein
(a) epcoritamab is administered subcutaneously
(i) at a priming dose of 0.16 mg on day 1 of the first 21-day cycle, at an intermediate dose of 0.8 mg on day 8 of the first 21-day cycle, and at a full dose of 48 mg on day 15 of the first 21-day cycle,
(ii) at a full dose of 48 mg on days 1, 8 and 15 of the second to fourth 21-day cycle, and
(iii) at a full dose of 48 mg on day 1 of each subsequent 21-day cycle;
(b) polatuzumab vedotin is administered at a dose of 1.8 mg/kg on day 1 of each 21-day cycle;
(c) rituximab is administered intravenously at a dose of 375 mg/m² on day 1 of each 21-day cycle;
(d) cyclophosphamide is administered intravenously at a dose of 750 mg/m² day 1 of each 21-day cycle;
(e) doxorubicin is administered intravenously at a dose of 50 mg/m² on day 1 of each 21-day cycle; and
(f) prednisone or prednisolone is administered intravenously or orally at a dose of 100 mg/day on days 1-5 of each 21-day cycle;
wherein administration of the combination in 21-day cycles continues until the subject exhibits a complete response (CR) or until progressive disease develops or unacceptable toxicity occurs.


SEQUENCE LISTING (29 sequences)

SEQ ID NO: 1 - protein, 8 aa, synthetic construct
  REGION 1..8: note = CDR sequence
GFTFNTYA

SEQ ID NO: 2 - protein, 10 aa, synthetic construct
  REGION 1..10: note = CDR sequence
IRSKYNNYAT

SEQ ID NO: 3 - protein, 16 aa, synthetic construct
  REGION 1..16: note = CDR sequence
VRHGNFGNSYVSWFAY

SEQ ID NO: 4 - protein, 9 aa, synthetic construct
  REGION 1..9: note = CDR sequence
TGAVTTSNY

SEQ ID NO: 5 - protein, 9 aa, synthetic construct
  REGION 1..9: note = CDR sequence
ALWYSNLWV

SEQ ID NO: 6 - protein, 125 aa, synthetic construct
  REGION 1..125: note = Heavy chain variable region sequence l
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 7 - protein, 109 aa, synthetic construct
  REGION 1..109: note = Light chain variable region sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 8 - protein, 8 aa, synthetic construct
  REGION 1..8: note = CDR sequence
GFTFHDYA

SEQ ID NO: 9 - protein, 8 aa, synthetic construct
  REGION 1..8: note = CDR sequence
ISWNSGTI

SEQ ID NO: 10 - protein, 15 aa, synthetic construct
  REGION 1..15: note = CDR sequence
AKDIQYGNYYYGMDV

SEQ ID NO: 11 - protein, 6 aa, synthetic construct
  REGION 1..6: note = CDR sequence
QSVSSY

SEQ ID NO: 12 - protein, 9 aa, synthetic construct
  REGION 1..9: note = CDR sequence
QQRSNWPIT

SEQ ID NO: 13 - protein, 122 aa, synthetic construct
  REGION 1..122: note = Heavy chain variable region sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 14 - protein, 107 aa, synthetic construct
  REGION 1..107: note = Light chain variable region sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 15 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 18 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 19 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 20 - protein, 329 aa, synthetic construct
  REGION 1..329: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 21 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 107 aa, Homo sapiens [sequence omitted]

SEQ ID NO: 24 - protein, 454 aa, synthetic construct
  REGION 1..454: note = Antibody heavy chain
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPG

SEQ ID NO: 25 - protein, 215 aa, synthetic construct
  REGION 1..215: note = Antibody light chain
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 451 aa, synthetic construct
  REGION 1..451: note = Antibody heavy chain
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPG

SEQ ID NO: 27 - protein, 214 aa, synthetic construct
  REGION 1..214: note = Antibody light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 29 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP